(19) United States
(12) Patent Application Publication
(10) Pub. No.: US 2025/0134853 A1
(43) Pub. Date: May 1, 2025

(54) COMPOUNDS AND METHODS FOR USING GALLOYLATED POLYPHENOLS TO TREAT DISEASES MEDIATED BY THIOL ISOMERASES

(71) Applicant: Beth Israel Deaconess Medical Center, Inc., Boston, MA (US)

(72) Inventors: Robert Flaumenhaft, Newton, MA (US); Moua Yang, Quincy, MA (US)

(21) Appl. No.: 18/934,169

(22) Filed: Oct. 31, 2024

Related U.S. Application Data
(60) Provisional application No. 63/594,542, filed on Oct. 31, 2023.

(51) Int. Cl. A61K 31/352; A61P 7/02

(52) U.S. Cl. CPC A61K 31/352; A61P 7/02

(57) ABSTRACT

Provided herein are galloylated polyphenols useful in inhibiting thrombosis formation. Also provided herein are methods of treating or preventing thrombosis in a subject in need thereof that include administering to the subject a galloylated polyphenol, or a pharmaceutically acceptable salt thereof.

### CROSS REFERENCE TO RELATED APPLICATIONS

This application claims priority to U.S. Application No. 63/594,542, filed on Oct. 31, 2023, the entire contents of which are hereby incorporated by reference.

### FEDERALLY SPONSORED RESEARCH OR DEVELOPMENT

This invention was made with government support under Grant Nos. HL135775 HL167383, HL143365, and HL164888 and awarded by the National Institutes of Health. The government has certain rights in the invention.

### TECHNICAL FIELD

The present disclosure relates to compounds such as galloylated polyphenols useful in inhibiting one or more thiol isomerases.

### BACKGROUND

Gallic acid is capable of free radical scavenging and galloylated polyphenols evolved, in part, to protect plants from oxidative stress and viral infection. Mammals lack the galloyltransferase required to synthesize galloylated polyphenols, so ingestion from plant sources represents the only means to acquire these compounds. Galloylated polyphenols are enriched in commonly consumed beverages such as tea and coffee and in high concentrations in dietary supplements. Antithrombotic effects have been noted among several potential cardiovascular protective activities. However, the identification of active constituents and the underlying mechanism of the antithrombotic activity of galloylated polyphenols action is largely unknown.

### SUMMARY

The present disclosure is based, at least in part, on the finding that galloylated polyphenols and compounds such as theaflavin, pinocembrin, catechin, and epigallocatechin (EGC), have activity against one or more thiol isomerases. In some embodiments, compounds of the present disclosure are anti-thrombotic agents. Advantageously, these compounds can be sourced from abundant agricultural products such as teas and are generally orally available.

Provided herein are methods of treating or preventing a thiol isomerase associated disease or disorder in a subject in need thereof, the method comprising administering to the subject a therapeutically effective amount of a compound provided herein (e.g., a galloylated polyphenol, or a pharmaceutically acceptable salt thereof). Also provided herein are methods of treating or preventing thrombosis in a subject in need thereof, the method comprising administering to the subject a therapeutically effective amount of a compound provided herein (e.g., a galloylated polyphenol, or a pharmaceutically acceptable salt thereof). Also provided herein are methods of inhibiting thrombus formation in a subject in need thereof, the method comprising administering to the subject a therapeutically effective amount of a compound provided herein (e.g., a galloylated polyphenol, or a pharmaceutically acceptable salt thereof). Also provided here are methods of inhibiting a thiol isomerase in a subject in need thereof, the method comprising administering to the subject a therapeutically effective amount of a compound provided herein (e.g., a galloylated polyphenol, or a pharmaceutically acceptable salt thereof). In some embodiments, the compounds is a galloylated polyphenol, or pharmaceutically acceptable salt thereof. In some embodiments, the galloylated polyphenol, or pharmaceutically acceptable salt thereof, is not pinocembrin 7-O-(3″-galloyl-4″,6″-(S)-hexahydroxydiphenoyl)-beta-D-glucose (PGHG) or epigallocatechin (EGC).

In some embodiments, the galloylated polyphenol, or pharmaceutically acceptable salt thereof, has a molecular weight of about 275 Da to about 5000 Da. In some embodiments, the galloylated polyphenol, or pharmaceutically acceptable salt thereof, has a molecular weight of about 600 Da to about 3000 Da. In some embodiments, the galloylated polyphenol, or pharmaceutically acceptable salt thereof, has a molecular weight of about 700 Da to about 1800 Da.

In some embodiments, the galloylated polyphenol, or pharmaceutically acceptable salt thereof, has a polar surface area of about 110 Å² to about 2000 Å². In some embodiments, the galloylated polyphenol, or pharmaceutically acceptable salt thereof, has a polar surface area of about 200 Å² to about 1500 Å². In some embodiments, the galloylated polyphenol, or pharmaceutically acceptable salt thereof, has a polar surface area of about 250 Å² to about 800 Å².

In some embodiments, the galloylated polyphenol, or pharmaceutically acceptable salt thereof, is selected from the group consisting of a galloylated theaflavin, a galloylated flavonone, a gallotannin, an ellagitannin, and a galloylated catechin, or a pharmaceutically acceptable salt thereof.

In some embodiments, the galloylated theaflavin, or pharmaceutically acceptable salt thereof, is selected from the group consisting of theaflavin-3-gallate, theaflavin-3′-gallate, and theaflavin-3-3′-digallate, or a pharmaceutically acceptable salt thereof.

In some embodiments, the gallotannin, or pharmaceutically acceptable salt thereof, is selected from the group consisting of pentagalloylglucose and gallic acid, or a pharmaceutically acceptable salt thereof.

In some embodiments, the ellagitannin, or pharmaceutically acceptable salt thereof, is selected from the group consisting of punicalagin, castalagin, vescalagin, and corilagin, or a pharmaceutically acceptable salt thereof.

In some embodiments, the galloylated catechin, or pharmaceutically acceptable salt thereof, is selected from the group consisting of epigallocatechin (EGC), epigallocatechin gallate (EGCG), and catechin 3-gallate, or a pharmaceutically acceptable salt thereof.

In some embodiments, the galloylated polyphenol, or pharmaceutically acceptable salt thereof, is selected from the group consisting of theaflavin 3-gallate, theaflavin 3,3′-digallate, pentagalloyl-glucose, gallic acid, punicalagin, EGCG, and catechin 3-gallate, or a pharmaceutically acceptable salt thereof. In some embodiments, the galloylated polyphenol, or pharmaceutically acceptable salt thereof, is selected from the group consisting of theaflavin 3-gallate, theaflavin 3,3′-digallate, and pentagalloyl-glucose, or a pharmaceutically acceptable salt thereof.

In some embodiments, the galloylated polyphenol, or pharmaceutically acceptable salt thereof, inhibits one or more thiol isomerases selected from the group consisting of PDI, ERp46, ERp57, ERp5, and ERp72. In some embodiments, the galloylated polyphenol, or pharmaceutically acceptable salt thereof, inhibits PDI, ERp57, and ERp5. In some embodiments, the galloylated polyphenol, or pharmaceutically acceptable salt thereof, inhibits ERp5.

In some embodiments, the galloylated polyphenol, or pharmaceutically acceptable salt thereof, inhibits at least one of the one or more thiol isomerases with an IC₅₀ of about 0.1 μM to about 50 μM. In some embodiments, the galloylated polyphenol, or pharmaceutically acceptable salt thereof, inhibits at least one of the one or more thiol isomerases with an IC₅₀ of about 0.1 μM to about 20 μM.

In some embodiments, the galloylated polyphenol, or pharmaceutically acceptable salt thereof, inhibits one or more thiol isomerases having a CGHC catalytic motif. In some embodiments, the galloylated polyphenol, or pharmaceutically acceptable salt thereof, reversibly inhibits one or more thiol isomerases.

In some embodiments, the galloylated polyphenol, or pharmaceutically acceptable salt thereof, is administered at a dosage of about 0.1 mg/kg to about 100 mg/kg.

In some embodiments, the galloylated polyphenol, or pharmaceutically acceptable salt thereof, inhibits fibrin formation in the subject. In some embodiments, the galloylated polyphenol, or pharmaceutically acceptable salt thereof, inhibits platelet accumulation in the subject. In some embodiments, the galloylated polyphenol, or pharmaceutically acceptable salt thereof, inhibits both fibrin formation and platelet accumulation in the subject.

In some embodiments, the subject in need thereof has venous thrombosis and/or arterial thrombosis. In some embodiments, the subject in need thereof has one or more of deep vein thrombosis, a pulmonary embolism, an arterial embolism, femoral vein thrombosis, Paget-Schroetter syndrome (PSS), myocardial infarction, superior vena cava thrombosis, jugular vein thrombosis, thrombotic stroke, cerebral venous sinus thrombosis, cavernous sinus thrombosis, retinal vein occlusion, May-Thurner syndrome, portal vein thrombosis, Budd-Chiari syndrome, and renal vein thrombosis.

In some embodiments, the subject in need thereof is at risk for thrombosis. In some embodiments, the subject in need thereof is at risk of venous thrombosis and/or arterial thrombosis. In some embodiments, the subject in need thereof is at risk of one or more of deep vein thrombosis, a pulmonary embolism, an arterial embolism, femoral vein thrombosis, Paget-Schroetter syndrome (PSS), myocardial infarction, superior vena cava thrombosis, jugular vein thrombosis, thrombotic stroke, cerebral venous sinus thrombosis, cavernous sinus thrombosis, retinal vein occlusion, May-Thurner syndrome, portal vein thrombosis, Budd-Chiari syndrome, and renal vein thrombosis.

In some embodiments, the subject at risk of thrombosis is a subject that has had one or more of a heart valve replacement, a mitral valve repair, and an orthopedic surgery. In some embodiments, the orthopedic surgery is a surgery that limits mobility. In some embodiments, the orthopedic surgery is a hip replacement surgery or a knee replacement surgery.

In some embodiments, the subject at risk of thrombosis is a subject that has previously been diagnosed with one or more of a blood clot, a blood clotting disorder, atrial fibrillation, and a cancer. In some embodiments, the blood clotting disorder is hemophilia, thrombophilia, Von Willebrand disease (VWD), Factor V Leiden, prothrombin gene mutation, antiphospholipid syndrome, and disseminated intravascular coagulation (DIC). In some embodiments, the cancer is pancreatic cancer, stomach cancer, lung cancer, colon cancer, kidney cancer, or multiple myeloma. In some embodiments, the subject at risk of thrombosis is a subject that has been immobilized for a prolonged period of time.

Also provided here are methods of treating or preventing thrombosis in a subject in need thereof, the method comprising administering to the subject a therapeutically effective amount of a galloylated polyphenol, or a pharmaceutically acceptable salt thereof, wherein the galloylated polyphenol is selected from the group consisting of theaflavin 3-gallate, theaflavin 3,3′-digallate, pentagalloyl-glucose, gallic acid, punicalagin, EGCG, catechin 3-gallate, or a combination thereof. In some embodiments, provided herein are methods of inhibiting thrombus formation in a subject in need thereof, the method comprising administering to the subject a therapeutically effective amount of a galloylated polyphenol, or a pharmaceutically acceptable salt thereof, wherein the galloylated polyphenol is selected from the group consisting of theaflavin 3-gallate, theaflavin 3,3′-digallate, pentagalloyl-glucose, gallic acid, punicalagin, EGCG, catechin 3-gallate, or a combination thereof. In some embodiments, provided herein are methods of inhibiting a thiol isomerase in a subject in need thereof, the method comprising administering to the subject a therapeutically effective amount of a galloylated polyphenol, or a pharmaceutically acceptable salt thereof, wherein the galloylated polyphenol is selected from the group consisting of theaflavin 3-gallate, theaflavin 3,3′-digallate, pentagalloyl-glucose, gallic acid, punicalagin, EGCG, catechin 3-gallate, or a combination thereof.

In some embodiments, the galloylated polyphenol, or pharmaceutically acceptable salt thereof, inhibits one or more thiol isomerases selected from the group consisting of PDI, ERp46, ERp57, ERp5, and ERp72. In some embodiments, the galloylated polyphenol, or pharmaceutically acceptable salt thereof, inhibits PDI, ERp57, and ERp5. In some embodiments, the galloylated polyphenol, or pharmaceutically acceptable salt thereof, inhibits ERp5.

In some embodiments, the galloylated polyphenol, or pharmaceutically acceptable salt thereof, inhibits at least one of the one or more thiol isomerases with an IC₅₀ of about 0.1 μM to about 50 μM. In some embodiments, the galloylated polyphenol, or pharmaceutically acceptable salt thereof, inhibits at least one of the one or more thiol isomerases with an IC₅₀ of about 0.1 μM to about 20 μM.

In some embodiments, the galloylated polyphenol, or pharmaceutically acceptable salt thereof, inhibits one or more thiol isomerases having a CGHC catalytic motif. In some embodiments, the galloylated polyphenol, or pharmaceutically acceptable salt thereof, reversibly inhibits one or more thiol isomerases.

In some embodiments, the galloylated polyphenol, or pharmaceutically acceptable salt thereof, is administered at a dosage of about 0.1 mg/kg to about 100 mg/kg.

In some embodiments, the galloylated polyphenol, or pharmaceutically acceptable salt thereof, inhibits fibrin formation in the subject. In some embodiments, the galloylated polyphenol, or pharmaceutically acceptable salt thereof, inhibits platelet accumulation in the subject. In some embodiments, the galloylated polyphenol, or pharmaceutically acceptable salt thereof, inhibits both fibrin formation and platelet accumulation in the subject.

In some embodiments, the subject in need thereof has venous thrombosis and/or arterial thrombosis. In some embodiments, the subject in need thereof has one or more of deep vein thrombosis, a pulmonary embolism, an arterial embolism, femoral vein thrombosis, Paget-Schroetter syndrome (PSS), myocardial infarction, superior vena cava thrombosis, jugular vein thrombosis, thrombotic stroke, cerebral venous sinus thrombosis, cavernous sinus thrombosis, retinal vein occlusion, May-Thurner syndrome, portal vein thrombosis, Budd-Chiari syndrome, and renal vein thrombosis.

In some embodiments, the subject in need thereof is at risk for thrombosis. In some embodiments, the subject in need thereof is at risk of venous thrombosis and/or arterial thrombosis. In some embodiments, the subject in need thereof is at risk of one or more of deep vein thrombosis, a pulmonary embolism, an arterial embolism, femoral vein thrombosis, Paget-Schroetter syndrome (PSS), myocardial infarction, superior vena cava thrombosis, jugular vein thrombosis, thrombotic stroke, cerebral venous sinus thrombosis, cavernous sinus thrombosis, retinal vein occlusion, May-Thurner syndrome, portal vein thrombosis, Budd-Chiari syndrome, and renal vein thrombosis.

In some embodiments, the subject at risk of thrombosis is a subject that has had one or more of a heart valve replacement, a mitral valve repair, and an orthopedic surgery. In some embodiments, the orthopedic surgery is a surgery that limits mobility. In some embodiments, the orthopedic surgery is a hip replacement surgery or a knee replacement surgery.

In some embodiments, the subject at risk of thrombosis is a subject that has previously been diagnosed with one or more of a blood clot, a blood clotting disorder, atrial fibrillation, and a cancer. In some embodiments, the blood clotting disorder is hemophilia, thrombophilia, Von Willebrand disease (VWD), Factor V Leiden, prothrombin gene mutation, antiphospholipid syndrome, and disseminated intravascular coagulation (DIC). In some embodiments, the cancer is pancreatic cancer, stomach cancer, lung cancer, colon cancer, kidney cancer, or multiple myeloma. In some embodiments, the subject at risk of thrombosis is a subject that has been immobilized for a prolonged period of time.

In some embodiments, the galloylated polyphenol, or pharmaceutically acceptable salt thereof, is theaflavin 3-gallate, or a pharmaceutically acceptable salt thereof. In some embodiments, the galloylated polyphenol, or pharmaceutically acceptable salt thereof, is theaflavin 3,3′-digallate, or a pharmaceutically acceptable salt thereof. In some embodiments, the galloylated polyphenol, or pharmaceutically acceptable salt thereof, tannic acid, or a pharmaceutically acceptable salt thereof. In some embodiments, the galloylated polyphenol, or pharmaceutically acceptable salt thereof, is pentagalloyl-glucose, or a pharmaceutically acceptable salt thereof. In some embodiments, the galloylated polyphenol, or pharmaceutically acceptable salt thereof, is gallic acid, or a pharmaceutically acceptable salt thereof. In some embodiments, the galloylated polyphenol, or pharmaceutically acceptable salt thereof, is punicalagin, or a pharmaceutically acceptable salt thereof. In some embodiments, the galloylated polyphenol, or pharmaceutically acceptable salt thereof, is EGCG, or a pharmaceutically acceptable salt thereof. In some embodiments, the galloylated polyphenol, or pharmaceutically acceptable salt thereof, is catechin 3-gallate, or a pharmaceutically acceptable salt thereof.

### Definitions

As used herein, “treating” or “treatment” refers to 1) inhibiting a disease; for example, inhibiting a disease, condition, or disorder in an individual who is experiencing or displaying the pathology or symptomatology of the disease, condition, or disorder (i.e., arresting further development of the pathology and/or symptomatology), or 2) ameliorating a disease; for example, ameliorating a disease, condition, or disorder in an individual who is experiencing or displaying the pathology or symptomatology of the disease, condition, or disorder (i.e., reversing the pathology and/or symptomatology).

As used herein, “preventing” or “prevention” of a disease, condition, or disorder refers to decreasing the risk of occurrence of the disease, condition, or disorder in a subject or group of subjects (e.g., a subject or group of subjects predisposed to or susceptible to the disease, condition or disorder). In some embodiments, preventing a disease, condition, or disorder refers to decreasing the possibility of acquiring the disease, condition, or disorder and/or its associated symptoms. In some embodiments, preventing a disease, condition, or disorder refers to completely or almost completely stopping the disease, condition, or disorder from occurring.

As used herein, the term “administration” can refer to the administration of a composition to a subject or system to achieve delivery of an active agent. Those of ordinary skill in the art will be aware of a variety of routes that may, in appropriate circumstances, be utilized for administration to a subject, for example a human. For example, in some embodiments, administration may be ocular, oral, parenteral, or topical.

In some embodiments, an active agent described herein can be administered orally. Examples of compositions suitable for oral administration include capsules, sachets, granules, tablets, powders, solutions or suspensions in a liquid, oil-in-water liquid emulsions, water-in-oil liquid emulsions, packed in liposomes, and boluses. In some embodiments, an active agent may be contained in a composition such that it is suitable for oral administration, for example, by combining the active agent with an inert diluent or an assimilable edible carrier.

As used herein, the terms “effective amount” and “effective to treat” can refer to an amount of a compound utilized for a period of time (e.g., acute or chronic administration, periodic or continuous administration) that is effective within the context of its administration for causing an intended effect or physiological outcome. Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen. Further, the dose to be administered can vary depending upon the age, weight, and general condition of the patient as well as the severity of the condition being treated, the judgment of the healthcare professional, and the particular mode of administration.

As used herein, the terms “subject” and “patient” are used interchangeably throughout the specification to describe an organism, typically a mammal, human or non-human, to whom treatment according to the methods of the present disclosure is provided. Veterinary applications are contemplated by the present disclosure. The terms include, but are not limited to, mammals, e.g., humans, other primates, pigs, hamsters, mice, rats, cows, horses, cats, dogs, sheep, and goats. In some embodiments, a subject is suffering from a relevant disease, disorder, or condition. In some embodiments, a subject is susceptible to a disease, disorder, or condition. In some embodiments, a subject displays one or more symptoms or characteristics of a disease, disorder, or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows intravital microscopy images of the cremasteric artery following intraperitoneal injection of vehicle or 25 mg/kg PGHG as indicated. The galloylated polyphenol PGHG inhibits platelet (red) and fibrin (green) accumulation in vivo in mice when injected intraperitoneally prior to injuring the cremasteric arterioles with a laser.

FIG. 2 shows the structures of the galloylated polyphenols that inhibit PDI in light gray, including PGHG, tannic acid, and theaflavin 3′3-digallate. Epigallocatechin gallate (EGCG) and the polyphenol backbones in dark gray (pinocembrin analog, gallic acid, theaflavin, and EGC) are inactive compounds against PDI, suggesting that galloylation must be correctly positioned within the enzyme for inhibition.

FIG. 3A is a plot showing PGHG inhibition of PDI. PDI was incubated with the indicated concentrations of PGHG for 15 minutes. PDI reductase activity was subsequently monitored using an insulin reductase assay. Data represented as mean±SEM.

FIG. 3B is a plot showing PGHG inhibition of PDI. PDI was incubated with the indicated concentrations of PGHG for 15 minutes. Reductase activity was then monitored by evaluating the ability of PDI to cleave a di-eosin-GSSG probe. Data represented as mean SEM.

FIG. 3C is a plot showing PGHG inhibition of PDI fragments. Isolated fragments of PDI including the a domain, an ab fragment, an abb′ fragment, a b′xa′ fragment, the a′ domain, and full-length PDI (abb′xa′) were evaluated for susceptibility to 10 μM PGHG using a di-eosin-GSSG assay. Data represented as mean±SEM.

FIG. 4 is a plot showing the binding of MPB to PDI in the presence of PGHG. The indicated concentrations of PGHG were incubated with PDI for 15 minutes. PDI was then exposed to MPB in Laemmli Sample buffer for 10 minutes. Samples were subsequently analyzed for MPB labeling with streptavidin-TRP after SDS-PAGE and immunoblotting.

FIG. 5 is an image of gel showing detection of PDI free thiol by the labeling of acetamido maleimidylstilbene disulfonic acid (AMS), which shifts the protein to a higher apparent molecular weight.

FIGS. 6A and 6B are plots showing % PDI activity. PDI was incubated with either 500 nM or 50 μM (FIG. 6A) NEM or (FIG. 6B) PGHG for 30 min, as indicated. To test reversibility, the samples containing 50 μM compounds were then diluted 1:100. PDI activity was evaluated using a di-eosin-GSSG reductase assay. Data represented as mean SEM.

FIG. 7A is a schematic from molecular docking of the binding between PGHG and the a domain of PDI shows interactions, including an interaction between His55 and a galloyl moiety of PGHG.

FIG. 7B is a schematic from molecular modeling of the binding of PGHG with the a domain of PDI showing His55 interaction with the galloyl groups of PGHG.

FIG. 8 is a plot showing % PDI activity. Wild-type PDI and PDI mutants in which His55 and His399 were replaced by alanine, aspartic acid, or arginine were incubated with 10 μM PGHG or vehicle for 15 min and evaluated using a di-eosin-GSSG reductase assay. Data represented as mean±SEM.

FIG. 9A is a plot showing inhibition of di-eosin-GSSG reductase activity of ERp57 at the the indicated concentrations of PGHG. Data represented as mean±SEM of at least 3 replicates.

FIG. 9B is a plot showing inhibition of di-eosin-GSSG reductase activity of ERp5 at the the indicated concentrations of PGHG. Data represented as mean±SEM of at least 3 replicates.

FIG. 9C is a plot showing inhibition of di-eosin-GSSG reductase activity of Erp72 at the the indicated concentrations of PGHG. Data represented as mean±SEM of at least 3 replicates.

FIG. 9D is a plot showing inhibition of di-eosin-GSSG reductase activity of ERp46 at the the indicated concentrations of PGHG. Data represented as mean±SEM of at least 3 replicates.

FIG. 10A is a plot showing median integrated fluorescence intensity over time of fibrin.

FIG. 10B is a plot showing quantification of the normalized fibrin accumulation as area under the curve (AUC) with the median shown. Vehicle, N=29 injuries; PGHG, N=27 injuries. P-values were determined by non-parametric one-way ANOVA with Dunnett's post hoc analysis.

FIG. 10C is a plot showing median integrated fluorescence intensity over time of platelets.

FIG. 10D is a plot showing quantification of the normalized platelet accumulation as area under the curve (AUC) with the median shown. Vehicle, N=29 injuries; PGHG, N=27 injuries. P-values were determined by non-parametric one-way ANOVA with Dunnett's post hoc analysis.

FIG. 10E is a plot showing length of individual injuries measured and compared for vehicle and 25 μg/g PGHG.

FIG. 11A shows intravital microscopy images of the cremasteric artery following intravenous injection of vehicle or 25 μg/g PGHG as indicated.

FIG. 11B is a plot showing median integrated fluorescence intensity over time of fibrin.

FIG. 11C is a plot showing quantification of the normalized fibrin accumulation as area under the curve (AUC) with the median. Vehicle, N=31 injuries; PGHG, N=31 injuries. P-values were determined by non-parametric one-way ANOVA with Dunnett's post hoc analysis.

FIG. 11D is a plot showing median integrated fluorescence intensity over time of platelets.

FIG. 11E is a plot showing quantification of the normalized platelet accumulation as area under the curve (AUC) with the median. Vehicle, N=31 injuries; PGHG, N=31 injuries. P-values were determined by non-parametric one-way ANOVA with Dunnett's post hoc analysis.

FIG. 12A is a plot showing the amount of time to cessation of bleeding following tail clip. Mice were exposed to intraperitoneal injection of either vehicle or 25 μg/g PGHG. Error bars represented as mean±SEM.

FIG. 12B is a plot showing a Kaplan-Meyer depiction of cessation of bleeding following tail clip.

FIG. 12C is a plot showing total volume of blood loss following tail clip. Error bars represented as mean±SEM.

FIG. 13A is a plot showing % thiol isomerase activity. 20 μM pinocembrin, catechin, or gallic acid were incubated with either PDI, ERp57, or ERp5 (shown from left to right for each compound). Following a 15 minute incubation, samples were evaluated for reductase activity using a di-eosin-GSSG assay.

FIG. 13B-13E are plots showing representative IC₅₀ curves of pinocembrin (FIG. 13B), D-glucose (FIG. 13C), gallic acid (FIG. 13D), and theaflavin (FIG. 13E) on PDI activity. IC₅₀s were determined by dose-response curve fitting in Graphpad Prism using a nonlinear regression [inhibitor] vs response mathematical modeling.

FIGS. 14A-14D are plots showing % thiol isomerase activity for the indicated theaflavins (FIG. 14A), the indicated gallotannins (FIG. 14B), punicalagin (FIG. 14C), and the indicated catechins (FIG. 14D). The indicated theaflavins, gallotannins, punicalagin, and catechins, (20 μM), were incubated with either PDI, ERp57, or ERp5 (shown from left to right for each compound). Following a 15 minute incubation, samples were evaluated for reductase activity using a di-eosin-GSSG assay. Data represent the percentage of activity in samples exposed to polyphenols compared to samples exposed to vehicle control as 100%. Data represented as mean±SD of 3 replicates. All statistical analyses were compared to vehicle control.

FIG. 15A is a plot showing % PDI activity for the indicated galloylated polyphenols. The indicated galloylated polyphenols (20 μM) were evaluated for their ability to inhibit reduction of disulfides within insulin. Data represent the percentage of activity in samples exposed to polyphenols compared to samples exposed to vehicle control as 100%. Data represented as mean±SD of at least 3 replicates. All statistical analyses were compared to vehicle control.

FIG. 15B is a plot showing % a′ domain activity for the indicated galloylated polyphenols. The indicated galloylated polyphenols (20 μM) were evaluated for their ability to inhibit the cleavage of di-eosin-GSSG mediated by the isolated a′ domain. Data represent the percentage of activity in samples exposed to polyphenols compared to samples exposed to vehicle control as 100%. Data represented as mean±SD of at least 3 replicates. All statistical analyses were compared to vehicle control.

FIG. 16A shows fluorescence microscopy images of carotid artery demonstrating progression of platelet accumulation (red) and vessel autofluorescence (green). Wildtype C57Bl/6J mice were exposed to 100 μg/g punicalagin via oral gavage (n=5) or vehicle (n=7). The carotid artery was surgically isolated, exposed to 10% w/v FeCl₃ for 3 min, and visualized by fluorescence microscopy. Time to occlusion was monitored.

FIG. 16B is a plot showing a Kaplan-Meyer depiction of time to occlusion following FeCl₃ exposure.

FIG. 16C is a plot showing the amount of time to cessation of bleeding following tail clip. Data represented as mean±SEM.

FIG. 16D is a plot showing a Kaplan-Meyer depiction of cessation of bleeding following tail clip.

FIG. 16E is a plot showing estimation of hemoglobin concentration from blood loss in the tail transection assay after punicalagin ingestion. Data represented as mean SEM.

FIG. 17A is a plot showing a representative IC₅₀ curve of theaflavin-3-gallate on PDI activity. IC₅₀s were determined by dose-response curve fitting in Graphpad Prism using a nonlinear regression [inhibitor] vs response mathematical modeling.

FIG. 17B is a plot showing a representative IC₅₀ curve of theaflavin-3,3-digallate on PDI activity. IC₅₀s were determined by dose-response curve fitting in Graphpad Prism using a nonlinear regression [inhibitor] vs response mathematical modeling.

FIG. 17C is a plot showing a representative IC₅₀ curve of 1,2,3,4,6-O-pentagalloylglucose on PDI activity. IC₅₀s were determined by dose-response curve fitting in Graphpad Prism using a nonlinear regression [inhibitor] vs response mathematical modeling.

FIG. 17D is a plot showing a representative IC₅₀ curve of tannic acid on PDI activity. IC₅₀s were determined by dose-response curve fitting in Graphpad Prism using a nonlinear regression [inhibitor] vs response mathematical modeling.

FIG. 17E is a plot showing a representative IC₅₀ curve of punicalagin on PDI activity. IC₅₀s were determined by dose-response curve fitting in Graphpad Prism using a nonlinear regression [inhibitor] vs response mathematical modeling.

### DETAILED DESCRIPTION

Dietary phenolics are abundant constituents in edible plants and have been credited with diverse cardiovascular benefits including antithrombotic activities (Iqbal I, et al. Molecules. 28, 2023; Rana A, et al. J. Food Biochem. 46:e14264, 2022). These dietary phenolics are modified by multiple transferases, such as glycosyltransferases, O-methyltransferases, acyltransferases, galloyltransferases, and others. See Hano C, Tungmunnithum D. Medicines (Basel). 7, 2020; Xiao J, Kai G. Crit. Rev. Food Sci. Nutr. 52:85-101, 2012. Galloylation is of particular interest with regard to cardiovascular effects since the galloyl group is redox active owing to its aromatic hydroxyl groups and ability to quench free radicals. See He H F. Front Nutr. 9:888892, 2022; Xiang Z, et al. Food Res Int. 180:114068, 2024. Although galloyl groups are oxidized during fermentation of black tea, galloylated polyphenols are abundant in green tea, which has been associated with reduced risk of stroke and coronary artery disease. See Shen L, et al. J. Zhejiang Univ. Sci. B. 13:652-62, 2012; Li X, et al. Heart. 103:783-9, 2017; Miller P E, et al. Am. J. Med. 130:188-97.e5, 2017; Mukamal K J, et al. Circulation. 105:2476-81, 2002; Kuriyama S, al. JAMA. 296:1255-65, 2006; Pang J, et al. Int. J. Cardiol. 202:967-74. 2016; and Teramoto M, et al. Stroke. 52:957-65, 2021. Consumption of several fruits with high galloylated polyphenolic content—berries, pomegranate, chestnuts—has been reported to be cardioprotective (Iqbal I, et al. Molecules. 28, 2023), although definitive, prospective trials are largely lacking. Most studies evaluating protective effects of galloylated polyphenols have focused on their antioxidant potential. See Iqbal I, et al. Molecules. 28, 2023.

The present disclosure is based, at least in part, on the finding that galloylated polyphenols and structurally similar compounds have activity against one or more thiol isomerases. Accordingly, provided herein are methods of inhibiting a thiol isomerase in a subject in need thereof, the method comprising administering to the subject a therapeutically effective amount of a compound provided herein. Without being bound by any particular theory, and as discussed in this disclosure, numerous peer-reviewed scientific publications provide credible evidence that thiol isomerases are implicated in the pathology of various disorders. In some embodiments, provided herein are methods of treating or preventing a disease or disorder associated with a thiol isomerase, the method comprising administering to the subject a therapeutically effective amount of a compound provided herein. Disorders associated with a thiol isomerase can include thrombosis, various types of cancers, neurodegenerative diseases, infectious diseases, autoimmune diseases, and metabolic disorders.

In some embodiments, a subject in need thereof has been identified or diagnosed as having a disease or disorder associated with a thiol isomerase (e.g., any of the types of a diseases or disorders associated with a thiol isomerase described herein). In some embodiments, a method provided herein includes identifying a subject as having a disease or disorder associated with a thiol isomerase prior to administration of a compound provided herein. A subject can be identified or diagnosed as having a disease or disorder associated with a thiol isomerase using any of the methods known in the art. In some embodiments, a method provided herein includes monitoring a disease or disorder associated with a thiol isomerase in the subject after administration of one or more dosages of a compound provided herein. A disease or disorder associated with a thiol isomerase can be monitored in a subject using any of the methods known in the art.

In some embodiments, compounds of the present disclosure are anti-thrombotic agents. Thiol isomerases released from platelets and endothelial cells during injury and inflammation can promote vascular thrombosis. Provided herein are methods of treating or preventing thrombosis in a subject in need thereof, the method comprising administering to the subject a therapeutically effective amount of a compound provided herein. In some embodiments, provided herein are methods of inhibiting thrombus formation in a subject in need thereof, the method comprising administering to the subject a therapeutically effective amount of a compound provided herein. In some embodiments, a compound provided herein is selected from a galloylated polyphenol (e.g., any of the galloylated polyphenols provided herein), theaflavin, pinocembrin, catechin, and epigallocatechin (EGC), or a pharmaceutically acceptable salt thereof. In some embodiments, a compound provided herein is compound that is structurally similar to a galloylated polyphenol provided herein.

A galloylated polyphenol as provided herein includes polyphenols with a galloyl moiety. Polyphenols includes compounds that have more than one hydroxyl group on an aromatic ring. Classes of polyphenols include phenolic acids, flavonoids, stilbenes, and lignans. Gallic acid (also known as 3,4,5-trihydroxybenzoic acid) is a redox reactive moiety that can be incorporated by plants into polyphenols via galloyltransferases.

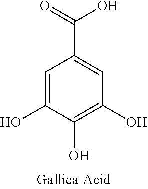 [SMILES: O=C(O)c1cc(O)c(O)c(O)c1]

Galloyltransferases are found in many plants and galloylated polyphenols are abundant in commonly consumed beverages such as teas. In some embodiments, a compound that is structurally similar to a galloylated polyphenol is a polyphenol having a trihydroxyphenyl moiety. In some embodiments, a compound that is structurally similar to a galloylated polyphenol is a polyphenol having a dihydroxyphenyl moiety or a phenol moiety. Galloylated polyphenols can include galloylated flavonoids, galloylated theaflavins, gallotannins or pentagalloylglucose, ellagitannins, and galloylated catechins.

Non-limiting examples of galloylated theaflavins include theaflavin-3-gallate, theaflavin-3′-gallate, and theaflavin-3-3′-digallate.

Non-limiting examples of gallotannins include tannic acid; pentagalloylglucose; digallic acid; gallic acid; galloyl glucoses (e.g., 1-galloyl glucose, glucogallin) such as digalloyl glucoses (e.g., 1,6-digalloyl glucose, 2,6-digalloyl glucose, 3,6-digalloyl glucose), trigalloyl glucoses (e.g., 1,2,3-trigalloyl glucose, 1,2,3-tri-O-galloyl-β-D-glucose, 1,2,6-trigalloyl glucose/1,3,6-trigalloyl glucose), tetragalloyl glucoses (e.g., 1,2,3,6-tetragalloylglucose, 1,2,3,6-tetra-O-galloyl-β-D-glucose, 1,2,4,6-tetragalloyl glucose, 1,2,4,6-tetra-O-galloyl-β-D-glucose), pentagalloyl glucoses (e.g., 1,2,3,4,6-pentagalloyl-glucose, 6-digalloyl-1,2,3-trigalloyl-glucose), and other galloyl glucoses (e.g., hexagalloyl glucose, heptagalloyl glucose, octagalloyl glucose, Nonagalloyl glucose, decagalloyl glucose); galloyl quinic acids such as 3-O-galloylquinic acid theogallin, 4-O-galloylquinic acid, 5-O-galloylquinic acid, digalloyl quinic acids (e.g., 1,4-di-O-galloylquinic acid, 3,4-di-O-galloylquinic acid, 3,5-di-O-galloylquinic acid, 4,5-di-O-Galloylquinic acid), and trigalloyl quinic acids (e.g., 1,3,4-tri-O-galloylquinic acid, 3,4,5-tri-O-galloylquinic acid); and galloyl shikimic acids (e.g., 4-O-galloyl shikimic acid, 5-O-galloyl shikimic acid, 3,5-di-O-galloyl-shikimic acid, 3,4,5-tri-O-galloylshikimic acid, 1,2,6-trigalloyl alloside, 1,3,6-trigalloyl alloside, 1,2,3,6-tetragalloyl alloside 1-O-galloyl proto-quercitol, 1,4-di-O-galloyl proto-quercitol).

Non-limiting examples of ellagitannins include punicalagin, castalagin, vescalagin, corilagin, castalin, casuarictin, grandinin, oenothein B, roburin A, tellimagrandin II, terflavin B, punicalin

Non-limiting examples of galloylated catechins include epigallocatechin (EGC), epigallocatechin gallate (EGCG), and catechin 3-gallate.

In some embodiments, a compound provided herein has a molecular weight of at least about 290 Da. For example, the compound can have a molecular weight of at least about 400 Da, at least about 600 Da, at least about 800 Da, at least about 1000 Da, at least about 1200 Da, at least about 1400 Da, at least about 1600 Da, at least about 2000 Da, at least about 2200 Da, at least about 2400 Da, at least about 2600 Da, at least about 3000 Da, at least about 3200 Da, at least about 3400 Da, at least about 3600 Da, at least about 3800 Da, at least about 4000 Da, at least about 4200 Da, at least about 4400 Da, at least about 4600 Da, or at least about 4800 Da.

Without wishing to be bound by any theory, the size of a compound may contribute to more potent inhibition of one or more thiol isomerases. In some embodiments, a compound provided herein, has a molecular weight of at least about 290 Da. For example, a compound provided herein can have a molecular weight of at least about 500 Da, at least about 700 Da, at least about 1000 Da, or at least about 1500 Da.

In some embodiments, a compound provided herein has a molecular weight of about 290 Da to about 5000 Da. For example, the compound can have a molecular weight of about 290 Da to about 600 Da, about 290 Da to about 1000 Da, about 290 Da to about 1400 Da, about 290 Da to about 1800 Da, about 290 Da to about 2200 Da, about 290 Da to about 2600 Da, about 290 Da to about 3000 Da, about 290 Da to about 3400 Da, about 290 Da to about 3800 Da, about 290 Da to about 4200 Da, about 290 Da to about 4600 Da, about 600 Da to about 1000 Da, about 600 Da to about 1400 Da, about 600 Da to about 1800 Da, about 600 Da to about 2200 Da, about 600 Da to about 2600 Da, about 600 Da to about 3000 Da, about 600 Da to about 3400 Da, about 600 Da to about 3800 Da, about 600 Da to about 4200 Da, about 600 Da to about 4600 Da, about 600 Da to about 5000 Da, about 1000 Da to about 1400 Da, about 1000 Da to about 1800 Da, about 1000 Da to about 2200 Da, about 1000 Da to about 2600 Da, about 1000 Da to about 3000 Da, about 1000 Da to about 3400 Da, about 1000 Da to about 3800 Da, about 1000 Da to about 4200 Da, about 1000 Da to about 4600 Da, about 1000 Da to about 5000 Da, about 1400 Da to about 1800 Da, about 1400 Da to about 2200 Da, about 1400 Da to about 2600 Da, about 1400 Da to about 3000 Da, about 1400 Da to about 3400 Da, about 1400 Da to about 3800 Da, about 1400 Da to about 4200 Da, about 1400 Da to about 4600 Da, about 1400 Da to about 5000 Da, about 1800 Da to about 2200 Da, about 1800 Da to about 2600 Da, about 1800 Da to about 3000 Da, about 1800 Da to about 3400 Da, about 1800 Da to about 3800 Da, about 1800 Da to about 4200 Da, about 1800 Da to about 4600 Da, about 1800 Da to about 5000 Da, about 2200 Da to about 2600 Da, about 2200 Da to about 3000 Da, about 2200 Da to about 3400 Da, about 2200 Da to about 3800 Da, about 2200 Da to about 4200 Da, about 2200 Da to about 4600 Da, about 2200 Da to about 5000 Da, about 2600 Da to about 3000 Da, about 2600 Da to about 3400 Da, about 2600 Da to about 3800 Da, about 2600 Da to about 4200 Da, about 2600 Da to about 4600 Da, about 2600 Da to about 5000 Da, about 3000 Da to about 3400 Da, about 3000 Da to about 3800 Da, about 3000 Da to about 4200 Da, about 3000 Da to about 4600 Da, about 3000 Da to about 5000 Da, about 3400 Da to about 3800 Da, about 3400 Da to about 4200 Da, about 3400 Da to about 4600 Da, about 3400 Da to about 5000 Da, about 3800 Da to about 4200 Da, about 3800 Da to about 4600 Da, about 3800 Da to about 5000 Da, about 4200 Da to about 4600 Da, about 4200 Da to about 5000 Da, or about 4800 Da to about 5000 Da.

Without wishing to be bound by any theory, the polar surface area of a compound may contribute to more potent inhibition of one or more thiol isomerases. In some embodiments, a compound provided herein has a polar surface area of at least about 110 Å². For example, the compound can have a polar surface area of at least about 250 Å², at least about 500 Å², at least about 750 Å², or at least about 1000 Å²).

In some embodiments, a compound provided herein has a polar surface area of about 110 Å² to about 2000 Å². For example, the compound can have a polar surface area of about 110 Å² to about 400 Å², about 110 Å² to about 600 Å², about 110 Å² to about 800 Å², about 110 Å² to about 1000 Å², about 110 Å² to about 1200 Å², about 110 Å² to about 1400 Å², about 110 Å² to about 1600 Å², about 110 Å² to about 1800 Å², about 250 Å² to about 400 Å², about 250 Å² to about 600 Å², about 250 Å² to about 800 Å², about 250 Å² to about 1000 Å², about 250 Å² to about 1200 Å², about 250 Å² to about 1400 Å², about 250 Å² to about 1600 Å², about 250 Å² to about 1800 Å², about 250 Å² to about 2000 Å², about 400 Å² to about 600 Å², about 400 Å² to about 800 Å², about 400 Å² to about 1000 Å², about 400 Å² to about 1200 Å², about 400 Å² to about 1400 Å², about 400 Å² to about 1600 Å², about 400 Å² to about 1800 Å², about 400 Å² to about 2000 Å², about 600 Å² to about 800 Å², about 600 Å² to about 1000 Å², about 600 Å² to about 1200 Å², about 600 Å² to about 1400 Å², about 600 Å² to about 1600 Å², about 600 Å² to about 1800 Å², about 600 Å² to about 2000 Å², about 800 Å² to about 1000 Å², about 800 Å² to about 1200 Å², about 800 Å² to about 1400 Å², about 800 Å² to about 1600 Å², about 800 Å² to about 1800 Å², about 800 Å² to about 2000 Å², about 1000 Å² to about 1200 Å², about 1000 Å² to about 1400 Å², about 1000 Å² to about 1600 Å², about 1000 Å² to about 1800 Å², about 1000 Å² to about 2000 Å², about 1200 Å² to about 1400 Å², about 1200 Å² to about 1600 Å², about 1200 Å² to about 1800 Å², about 1200 Å² to about 2000 Å², about 1400 Å² to about 1600 Å², about 1400 Å² to about 1800 Å², about 1400 Å² to about 2000 Å², about 1600 Å² to about 1800 Å², about 1600 Å² to about 2000 Å², or about 1800 Å² to about 2000 Å².

In some embodiments, a compound provided herein is a galloylated polyphenol, or pharmaceutically acceptable salt thereof. In some embodiments, the galloylated polyphenol, or pharmaceutically acceptable salt thereof, is selected from the group consisting of theaflavin 3-gallate, theaflavin 3,3′-digallate, pentagalloyl-glucose, gallic acid, punicalagin, EGCG, and catechin 3-gallate, or a pharmaceutically acceptable salt thereof.

In some embodiments, the galloylated polyphenol, or pharmaceutically acceptable salt thereof, is selected from the group consisting of theaflavin 3-gallate, theaflavin 3,3′-digallate, and pentagalloyl-glucose, or a pharmaceutically acceptable salt thereof.

In some embodiments, the galloylated polyphenol is not a galloylated polyphenol disclosed in U.S. Publication No. 2024/0016777 or International Publication No. WO2022/104153, each of which is incorporated herein by reference in its entirety. In some embodiments, the galloylated polyphenol, or pharmaceutically acceptable salt thereof, is not a flavonoid. Non-limiting examples of flavonoids include flavones, flavonols, flavanols, flavanones, isoflavones, proanthocyanidins, and anthocyanins.

In some embodiments, the galloylated polyphenol, or pharmaceutically acceptable salt thereof, is not pinocembrin 7-O-(3″-galloyl-4″,6″-(S)-hexahydroxydiphenoyl)-beta-D-glucose (PGHG).

### Thiol Isomerases

Thiol isomerases are endoplasmic reticulum (ER) resident proteins that bind nascent polypeptides and promote folding and optimal disulfide bond formation (Flaumenhaft R. Trends Cardiovasc Med. 23:264-8, 2013; Flaumenhaft R. Curr. Opin. Hematol. 24:439-45, 2017; Essex D W, Wang L. J. Thromb. Haemost. 2024). Thiol isomerases also play key roles in maintaining cellular homeostasis by catalyzing disulfide bond breakage, formation, and rearrangement. See, e.g., Gelzinis et al. FASEB J. 37(5):e22914, 2023.

Upon cell activation or injury, thiol isomerases can escape the confines of the ER and be released into the extracellular environment. Thiol isomerases are released from platelets and endothelial cells during injury and inflammation promote vascular thrombosis (Sharda A V, et al. Clin Cancer Res. 27:5708-17, 2021; Schulman S, et al. Antioxid Redox Signal. 24:1-15, 2016; Sharda A, et al. Blood. 125:1633-42. 2015; Cho J, et al. J Clin Invest. 118: 1123-31, 2008; Kim K, et al. Blood. 122: 1052-61, 2013; Oliveira P V S, et al. Redox Biol. 22:101142. 2019).

Protein disulfide isomerase (PDI), ERp5, ERp57, ERp72, and ERp46 are among the released thiol isomerases that have been shown to function in thrombus formation (Cho J, et al. J Clin Invest. 118: 1123-31, 2008; Wu Y, et al. Blood. 119: 1737-46, 2012; Zhou J, et al. Blood. 139: 2050-65, 2022; Zhou J, et al. J Clin Invest. 125:4391-406, 2015; Passam F H, et al. Blood. 125:2276-85, 2015; Holbrook L M, et al. J Thromb Haemost. 16:367-77, 2018; Reinhardt C, et al. J Clin Invest. 118:1110-22, 2008). Use of antibodies directed at specific thiol isomerases to inhibit thrombus formation has demonstrated that extracellular thiol isomerases mediate the prothrombotic activity of thiol isomerases (Cho J, et al. J Clin Invest. 118: 1123-31, 2008; Passam F H, et al. Blood. 125:2276-85, 2015; Holbrook L M, et al. J Thromb Haemost. 16:367-77, 2018; Reinhardt C, et al. J Clin Invest. 118:1110-22, 2008). Thiol isomerases mediate cysteine modifications including disulfide bond formation, disulfide bond cleavage, cysteine sulfenylation and cysteine nitrosation (see Hatahet F, Ruddock L W. Antioxidants & redox signaling. 11:2807-50, 2009; Yang M, et al. J Thromb Haemost. 21:2137-50, 2023; Bekendam R H, et al. J Thromb Haemost. 16:2322-35, 2018). However, thiol isomerase-mediated substrate and cysteine modifications that promote thrombosis in vivo have not been definitively characterized.

Compounds of the present disclosure can inhibit one or more thiol isomerases. Protein disulfide isomerase (PDI) family thiol isomerases are multidomain oxidoreductases. PDI has a domain structure of a-b-b′-a′ in which the a and a′ domains contain a catalytic site motif Cys-X-X-Cys (CXXC, where X is any amino acid) responsible for disulfide shuffling, and the b and b′ domains are the substrate binding domains. In some embodiments, a thiol isomerase contains a Cys-Gly-His-Cys catalytic site motif. In some embodiments, a compound provided herein inhibits one or more thiol isomerases having a CGHC catalytic motif. Non-limiting examples of thiol isomerases include PDI, ERp46, ERp57, ERp5, ERp72, ERp44, TMX4, and ERp29.

In some embodiments, provided herein are methods of inhibiting a thiol isomerase in a subject in need thereof, the method comprising administering to the subject a therapeutically effective amount of a compound provided herein. In some embodiments, the subject has any of the diseases or disorders described herein. For example, the subject can have thrombosis, a cancer (e.g., any of the cancers described herein), a neurodegenerative disorder (e.g., any of the neurodegenerative disorders described herein), an infectious disease (e.g., any of the infectious disorders described herein), an autoimmune disorder (e.g., any of the autoimmune disorders described herein), or a metabolic disorder (e.g., any of the metabolic disorders described herein). In some embodiments, the subject is at risk of thrombosis. In some embodiments, the method further comprises treating and/or preventing a thiol isomerase associated disease or disorder (e.g., any of the thiol isomerase associated diseases or disorders described herein).

In some embodiments, a compound provided herein can inhibit two or more thiol isomerases. For example, some compounds provided herein can inhibit PDI, ERp57, and ERp5. In some embodiments, compound provided herein can selectively inhibit one or more thiol isomerases or inhibit many or all thiol isomerases. In some embodiments, a compound provided herein can inhibit ERp5. In some embodiments, a compound provided herein can inhibit ERp5 more potently than other thiol isomerases.

In some embodiments, a compound provided herein inhibits at least one thiol isomerase with an IC₅₀ of less than about 50 μM. For example, the compound can inhibit at least one thiol isomerase with an IC₅₀ of less than about 40 μM, less than about 30 μM, less than about 25 μM, less than about 20 μM, less than about 15 μM, less than about 10 μM, or less than about 5 μM.

In some embodiments, a compound provided herein inhibits at least one thiol isomerase with an IC₅₀ of about 0.1 μM to about 50 μM. For example, the compound can inhibit at least one thiol isomerase with an IC₅₀ of about 0.1 μM to about 0.5 μM, about 0.1 μM to about 1 μM, about 0.1 μM to about 5 μM, about 0.1 μM to about 10 μM, about 0.1 μM to about 15 μM, about 0.1 μM to about 20 μM, about 0.1 μM to about 25 μM, about 0.1 μM to about 30 μM, about 0.1 μM to about 35 μM, about 0.1 μM to about 40 μM, about 0.5 μM to about 1 μM, about 0.5 μM to about 5 μM, about 0.5 μM to about 10 μM, about 0.5 μM to about 15 μM, about 0.5 μM to about 20 μM, about 0.5 μM to about 25 μM, about 0.5 μM to about 30 μM, about 0.5 μM to about 35 μM, about 0.5 μM to about 40 μM, about 0.5 μM to about 50 μM, about 1 μM to about 5 μM, about 1 μM to about 10 μM, about 1 μM to about 15 μM, about 1 μM to about 20 μM, about 1 μM to about 25 μM, about 1 μM to about 30 μM, about 1 μM to about 35 μM, about 1 μM to about 40 μM, about 1 μM to about 50 μM, about 5 μM to about 10 μM, about 5 μM to about 15 μM, about 5 μM to about 20 μM, about 5 μM to about 25 μM, about 5 μM to about 30 μM, about 5 μM to about 35 μM, about 5 μM to about 40 μM, about 5 μM to about 50 μM, about 10 μM to about 15 μM, about 10 μM to about 20 μM, about 10 μM to about 25 μM, about 10 μM to about 30 μM, about 10 μM to about 35 μM, about 10 μM to about 40 μM, about 10 μM to about 50 μM, about 15 μM to about 20 μM, about 15 μM to about 25 μM, about 15 μM to about 30 μM, about 15 μM to about 35 μM, about 15 μM to about 40 μM, about 15 μM to about 50 μM, about 20 μM to about 25 μM, about 20 μM to about 30 μM, about 20 μM to about 35 μM, about 20 μM to about 40 μM, about 20 μM to about 50 μM, about 25 μM to about 30 μM, about 25 μM to about 35 μM, about 25 μM to about 40 μM, about 25 μM to about 50 μM, about 30 μM to about 35 μM, about 30 μM to about 40 μM, about 30 μM to about 50 μM, about 35 μM to about 40 μM, about 35 μM to about 50 μM, or about 40 μM to about 50 μM.

Inhibition of the catalytic domains of PDI by a compound provided herein (e.g., galloylated polyphenol, or pharmaceutically acceptable salt thereof) could occur via a covalent interaction involving the active site cysteines or by a reversible mechanism. In some embodiments, a compound provided herein reversibly inhibits one or more thiol isomerases.

### Thrombosis

Thrombosis is the formation of a blood clot (thrombus) within blood vessels that can limit the natural flow of blood. Thrombosis can occur within venous blood vessels (venous thrombosis) and/or arterial blood vessels (arterial thrombosis). Acute venous and arterial thromboses are among the most common causes of death worldwide. As used herein, thrombosis also includes embolisms. For example, an embolism can occur when a thrombus dislodges (also referred to as an embolus) and creates a blockage in a different location of the blood vessel or in a different blood vessel. Non-limiting examples of thrombosis include deep vein thrombosis, a pulmonary embolism, an arterial embolism, femoral vein thrombosis, Paget-Schroetter syndrome (PSS), myocardial infarction, superior vena cava thrombosis, jugular vein thrombosis, thrombotic stroke, cerebral embolism, cerebral venous sinus thrombosis, cavernous sinus thrombosis, retinal vein occlusion, May-Thurner syndrome, portal vein thrombosis, Budd-Chiari syndrome, and renal vein thrombosis.

Components of a thrombus include aggregated platelets and red blood cells that form a plug, and a mesh of cross-linked fibrin protein. Anti-thrombotic agents can be classified as anticoagulant agents and antiplatelet agents. Anticoagulants can slow down clotting, thereby reducing fibrin formation and preventing clots from forming and growing. Antiplatelet agents can prevent platelets from clumping and also prevent clots from forming and growing. Anticoagulant agents and antiplatelet agents are used for both the treatment and prevention of thrombosis with antiplatelet agents being more commonly used for atherothrombosis and anticoagulants for venous thromboembolism. See, e.g., Chan & Weitz, Circ. Res. 124(3):426-436, 2019. In some embodiments, a compound provided herein inhibits fibrin formation and/or platelet accumulation in a subject.

In some embodiments, a subject in need thereof has been identified or diagnosed as having thrombosis (e.g., any of the types of thrombosis described herein). In some embodiments, a method provided herein includes identifying a subject as having thrombosis prior to administration of a compound provided herein. A subject can be identified or diagnosed as having thrombosis using any of the methods known in the art. For example, a subject can be identified or diagnosed as having thrombosis through the use of an ultrasound (e.g., venous ultrasound, duplex ultrasonography, Doppler ultrasound) a D-dimer blood test, contrast venography, magnetic resonance imaging, computed tomographic pulmonary angiography (CTPA), a ventilation-perfusion (V/Q) scan, and pulmonary angiography.

In some embodiments, a method provided herein includes monitoring thrombosis in the subject after administration of one or more dosages of a compound provided herein. For example, methods for identifying a blood clot (e.g., any of the methods for diagnosing thrombosis described herein) can be performed at multiple times during a course of monitoring and/or a course of treatment to determine one or more clinically relevant parameters including, without limitation, progression of the disease and efficacy of treatment with of a compound provided herein. In some embodiments, the size and/or presence of a blood clot can be monitored using, e.g., an ultrasound (e.g., venous ultrasound, duplex ultrasonography, Doppler ultrasound) contrast venography, magnetic resonance imaging, computed tomographic pulmonary angiography (CTPA), a ventilation-perfusion (V/Q) scan, and pulmonary angiography. A decrease in the size and/or dissolution of a blood clot during a course of treatment with a compound provided herein can indicate treatment of the thrombosis. In some embodiments, symptoms associated with thrombosis can be used to determine treatment efficacy during a course of treatment. For example, a decrease in symptoms associated with thrombosis during a course of treatment with a compound provided herein can indicate treatment of the thrombosis. Non-limiting examples of symptoms associated with thrombosis can include pain, swelling, tenderness, and/or warmth in a localized area (e.g., a leg, an arm, the abdomen), numbness on one side of the body, chest pain, coughing (including a cough that produces bloody mucus), dizziness, heart palpitations, shortness of breath that worsens with exertion, loss of function of a limb, facial droop, and slurred speech.

In some embodiments, a subject in need thereof is a subject at risk for thrombosis (e.g., any of the types of thrombosis described herein). There are many factors that are known to increase the risk of thrombosis. For example, major surgery and hospitalizations; heart conditions such as heart attack or congestive heart failure; chronic conditions including high blood pressure, diabetes, and kidney disease; lower-extremity paralysis due to spinal cord injury; fracture of the pelvis, hip or long bones; infections, such as the virus that causes COVID-19; multiple traumas; and cancers are all known to increase an individual's risk of thrombosis. See, e.g., Pastori et al. Int. J. Mol Sci. 24(4):3169, 2023; Kim et al. Transl Res. 225:33-53, 2020.

In some embodiments, a subject at risk of thrombosis is a subject that has had one or more of a heart valve replacement, a mitral valve repair, and an orthopedic surgery. In some embodiments, the orthopedic surgery is a surgery that limits mobility. In some embodiments, the orthopedic surgery is a hip replacement surgery or a knee replacement surgery.

In some embodiments, a subject at risk of thrombosis is a subject that has previously been diagnosed with one or more of a blood clot, a blood clotting disorder, atrial fibrillation, and a cancer. Non-limiting examples of blood clotting disorders include hemophilia, thrombophilia, Von Willebrand disease (VWD), Factor V Leiden, prothrombin gene mutation, antiphospholipid syndrome, and disseminated intravascular coagulation (DIC). Non-limiting examples of cancers that increase the risk of thrombosis include pancreatic cancer, stomach cancer, lung cancer, colon cancer, kidney cancer, or multiple myeloma. In some embodiments, a cancer that increases the risk of thrombosis is an advanced and/or metastatic cancer.

Prolonged immobilization, including transient immobility, can also increase the risk of thrombosis. See, e.g., Engbers et al. J. Thromb. Haemost. 12(3):290-6, 2014. For example, long flights can increase the risk of thrombosis with flights lasting about 8 to 10 hours or longer posing the greatest risk for thrombosis such as deep vein thrombosis. In some embodiments, immobilization for a period of about 3 or more days can increase the risk of thrombosis in a subject. For example, a prolonged period of immobilization of about 4, about 5, about 10, about 15, about 20, or about 45 or more days can increase the risk of thrombosis in a subject.

In some embodiments, a method provided herein includes identifying a subject as at risk of thrombosis prior to administration of a compound provided herein where the subject has one or more of the risk factors described herein. For example, prior to administration of a compound provided herein a method provided herein can include identifying or diagnosing a subject as having one or more of a prior history of blood clot(s), a blood clotting disorder, a prior history of atrial fibrillation, a cancer, a heart valve replacement, a mitral valve repair, an orthopedic surgery, and/or prolonged immobilization.

### Neurodegenerative Disorders

In some embodiments, compounds of the present disclosure can be useful in the prevention and/or treatment of neurodegenerative disorders. Neurodegeneration is often featured by accumulation in brain cells and intracellular spaces of insoluble protein aggregates, such as α-synuclein fibrils, amyloid-β plaques, and tau tangles, as well as by marked neuroinflammation. Together, these pathologies can lead to a reduction of brain volume and brain cell number, degeneration of neurons, dysfunction of microglia, and the development of various neurodegenerative disorders. Scientific publications attest to the well-established principle that modulating (e.g., inhibiting) one or more thiol isomerases in cells (e.g., brain cells) is an art-accepted mechanism for preventing and/or treating neurodegenerative disorders. See, e.g., Gonzalez-Perez et al. Gene. 566, 158-165, 2015; Hettinghouse A, et al. Pharmacol Ther. 181:34-48, 2018; which are incorporated herein by reference in their entireties.

Accordingly, provided herein are methods of treating or preventing a neurodegenerative disorder associated with one or more thiol isomerases in a subject, the method comprising administering to the subject a therapeutically effective amount of a compound provided herein (e.g., a galloylated polyphenol, or a pharmaceutically acceptable salt thereof). Suitable examples of neurodegenerative disorders include synucleinopathies, amyloidopathies, and/or tauopathies. For example, synucleinopathies include dementia with Lewy bodies, Parkinson's disease (PD), multiple system atrophy (MSA), pure autonomic failure (PAF), PD with dementia, olivopontocerebellar atrophy (OPCA), striatonigral degeneration (SND), neuroaxonal dystrophy, Shy-Drager syndrome, Hallervorden-Spatz syndrome, and Bradbury-Eggleston syndrome. Suitable examples of amyloidopathies include premature aging, cerebral amyloid angiopathy, Alzheimer's disease (AD), familial AD (FAD), dementia associated with AD or FAD, frontotemporal lobar degeneration (FTD), dementia associated with FTD, amyotrophic lateral sclerosis (ALS, aka Leu Gehrig's disease), Huntington's disease (HD), and dementia associated with HD. Suitable examples of tauopathies include Pick's disease, progressive supranuclear palsy, corticobasal degeneration, argyrophilic grain disease, primary age-related tauopathy, neurofibrillary tangle dementia, chronic traumatic encephalopathy (CTE), aging-related tau astrogliopathy, Richardson syndrome, cerebellar ataxia, globular glial tauopathy, and argyrophilic grain disease. In some embodiments, the neurodegenerative disorder associated with one or more thiol isomerases is selected from frontotemporal dementia, amyotrophic lateral sclerosis, Alzheimer's disease, peripheral nerve injury, a prion-protein related disorder, and Huntington's Disease.

### Metabolic and Cardiovascular Disorders

In some embodiments, compounds of the present disclosure can be useful in the treatment of metabolic and cardiovascular disorders. Examples of metabolic and cardiovascular disorders include ischemic myocardial injury, atherosclerosis, ischemic cardiomyopathy, coronary artery disease, type 1 diabetes, type 2 diabetes, hyperglycerimia, hypermethioninemia, familial hypercholesterolemia, Gaucher disease, Hunter syndrome, Krabbe disease, maple syrup urine disease, metachromatic leukodystrophy, mitochondrial encephalopathy, lactic acidosis, stroke-like episodes (MELAS), Niemann-Pick, phenylketonuria (PKU), porphyria, Tay-Sachs disease, and Wilson's disease.

Scientific publications attest to the well-established principle that modulating (e.g., inhibiting) one or more thiol isomerases in cells is an art-accepted mechanism for preventing and/or treating metabolic and/or cardiovascular disorders. See, e.g., Grek & Townsend, Endoplasmic Reticulum Stress Dis, 1(1):4-17, 2014; Jiang et al. Antioxid Redox Signal. 2022 7; 37(16):1191-1205, which are incorporated herein by reference in their entireties. Accordingly, provided herein are methods of treating or preventing a metabolic disorder associated with one or more thiol isomerases in a subject, the method comprising administering to the subject a therapeutically effective amount of a compound provided herein (e.g., a galloylated polyphenol, or a pharmaceutically acceptable salt thereof). In some embodiments, the metabolic or cardiovascular disorder associated with one or more thiol isomerases is selected from ischemic myocardial injury, atherosclerosis, ischemic cardiomyopathy, coronary artery disease, and diabetes.

### Autoimmune Disorders

In some embodiments, compounds of the present disclosure can be useful in the treatment of autoimmune disorders. Scientific publications attest to the well-established principle that modulating (e.g., inhibiting) one or more thiol isomerases in cells is an art-accepted mechanism for preventing and/or treating autoimmune disorders. See, e.g., Krajewski D, et al. Front Immunol. 11, 606837, 2020; J Immunol (2017) 198 (1_Supplement): 217.12; Int Immunopharmacol. 2020, 12:82:106286; which are incorporated herein by reference in their entireties. Accordingly, provided herein are methods of treating or preventing an autoimmune disorder associated with one or more thiol isomerases in a subject, the method comprising administering to the subject a therapeutically effective amount of a compound provided herein (e.g., a galloylated polyphenol, or a pharmaceutically acceptable salt thereof). Non-limiting examples of an autoimmune disorder include arthritis, autoimmune encephalitis, autoimmune thyroiditis, glomerulonephritis, necrotizing vasculitis, lymphadenitis, periarteritis nodosa, systemic lupus erythematosis, systemic sclerosis, dermatomyositis/polymyositis, anti-phospholipid antibody syndrome, scleroderma, pemphigus vulgaris, uveitis, Reiter's syndrome, ankylosing spondylitis, Lyme disease, Guillain-Barre syndrome, and cardiomyopathy.

### Cancers

In some embodiments, compounds of the present disclosure can be useful in the treatment of cancer. Scientific publications attest to the well-established principle that modulating (e.g., inhibiting) one or more thiol isomerases in cells (e.g., cancer cells) is an art-accepted mechanism for preventing and/or treating various cancer types. See, e.g., Gelzinis et al. FASEB J. 37(5):e22914, 2023; Xu S, et al. PNAS. 109, 16348-16353, 2012; Sharda A V, et al. Clinical Cancer Research. 27, 5708-5717, 2021; Parakh & Atkin, Front Cell Dev Biol. 21:3:30, 2015; which are incorporated herein by reference in their entireties.

Accordingly, provided herein are methods of treating or preventing a cancer associated with one or more thiol isomerases in a subject, the method comprising administering to the subject a therapeutically effective amount of a compound provided herein (e.g., a galloylated polyphenol, or a pharmaceutically acceptable salt thereof). Non-limiting examples of cancers associated with one or more thiol isomerases include cervical cancer, gastric mucosa cancer, laryngeal cancer, ovarian cancer, breast cancer, melanoma, lymphoma, leukemia, glioma, kidney, lung, brain, prostrate, and male germ cell tumors and colon cancer. In some embodiments, a cancer associated with one or more thiol isomerases is a cancer that overexpresses one or more thiol isomerases.

Additional exemplary cancers include acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer), brain cancer (e.g., meningioma, glioblastomas, glioma (e.g., astrocytoma, oligodendroglioma), medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), connective tissue cancer, epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, ocular cancer (e.g., intraocular melanoma, retinoblastoma), familiar hypereosinophilia, gall bladder cancer, gastric cancer (e.g., stomach adenocarcinoma), gastrointestinal stromal tumor (GIST), germ cell cancer, head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)), heavy chain disease (e.g., alpha chain disease, gamma chain disease, mu chain disease, hemangioblastoma, hypopharynx cancer, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), muscle cancer, myelodysplastic syndrome (MDS), mesothelioma, myeloproliferative disorder (MPD) (e.g., polycythemia vera (PV), essential thrombocytosis (ET), agnogenic myeloid metaplasia (AMM) aka. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma (e.g., bone cancer), ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), islet cell tumors), penile cancer (e.g., Paget's disease of the penis and scrotum), pinealoma, primitive neuroectodermal tumor (PNT), plasma cell neoplasia, paraneoplastic syndromes, intraepithelial neoplasms, prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), eposarcoma, malignant peripheral nerve sheath tumor (MPNST), chondrosarcoma, fibrosarcoma, myxosarcoma), sebaceous gland carcinoma, small intestine cancer, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer, and vulvar cancer (e.g., Paget's disease of the vulva).

### Infectious Diseases

In some embodiments, compounds of the present disclosure can be useful in the prevention and/or treatment of infectious diseases. For example, compounds provided herein have been shown to inhibit viral replication. Provided herein are methods of treating or preventing an infectious diseases associated with one or more thiol isomerases in a subject, the method comprising administering to the subject a therapeutically effective amount of a compound provided herein (e.g., a galloylated polyphenol, or a pharmaceutically acceptable salt thereof). In some embodiments, the infectious disease is caused by a coronavirus selected from SARS-CoV, SAR-CoV-2, and MERS-CoV. In some embodiments, the infectious disease is coronavirus disease 2019 (COVID-19). In some embodiments, the infectious disease is severe acute respiratory syndrome (SARS). In some embodiments, the infectious disease is Middle East respiratory syndrome (MERS).

In some embodiments, the method further comprises administering to the subject a therapeutically effective amount of an antiviral agent, or a pharmaceutically acceptable salt thereof.

### Pharmaceutical Compositions and Dosages

Also provided herein are pharmaceutical compositions which contain, as the active ingredient, an effective amount of a compound provided herein in combination with one or more pharmaceutically acceptable carriers (excipients).

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain. Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The pharmaceutical compositions provided herein can contain one or more of the compounds described herein in the range of 0.005% to 100% w/w with the balance made up from the suitable pharmaceutically acceptable excipients. For example, pharmaceutical compositions or dosage forms provided herein can contain about 0.001% to 100%, about 0.1% to about 95%, about 75 to about 85%, about 20% to about 80% w/w of one or more of the compounds provided herein, with the balance of the pharmaceutical composition made up of any pharmaceutically acceptable excipient described herein, or any combination of these excipients.

In some embodiments, the pharmaceutical compositions provided herein can contain about 100 mg to 1000 mg of one or more of the compounds described herein. For example, the pharmaceutical compositions provided herein can contain about 100 mg to 900 mg, about 100 mg to 800 mg, about 100 mg to 700 mg, about 100 mg to 600 mg, about 100 mg to 500 mg, about 100 mg to 400 mg, about 100 mg to 300 mg, about 100 mg to 200 mg, about 200 mg to 1000 mg, about 200 mg to 900 mg, about 200 mg to 800 mg, about 200 mg to 700 mg, about 200 mg to 600 mg, about 200 mg to 500 mg, about 200 mg to 400 mg, about 200 mg to 300 mg, about 200 mg to 1000 mg, about 300 mg to 900 mg, about 300 mg to 800 mg, about 300 mg to 700 mg, about 300 mg to 600 mg, about 300 mg to 500 mg, about 300 mg to 400 mg, about 400 mg to 1000 mg, about 400 mg to 900 mg, about 400 mg to 800 mg, about 400 mg to 700 mg, about 400 mg to 600 mg, about 400 mg to 500 mg, about 500 mg to 1000 mg, about 500 mg to 900 mg, about 500 mg to 800 mg, about 500 mg to 700 mg, about 500 mg to 600 mg, about 600 mg to 1000 mg, about 600 mg to 900 mg, about 600 mg to 800 mg, about 600 mg to 700 mg, about 700 mg to 1000 mg, about 700 mg to 900 mg, about 700 mg to 800 mg, about 800 mg to 1000 mg, about 800 mg to 900 mg, or about 900 mg to 1000 mg of one or more of the compounds described herein.

Compounds provided herein can be present in a pharmaceutical composition in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of a compound described herein, can range, for example, from about 0.1 mg/kg to about 100 mg/kg (e.g., about 0.1 mg/kg to about 75 mg/kg, about 0.1 mg/kg to about 50 mg/kg, about 0.1 mg/kg to about 25 mg/kg about 0.1 mg/kg to about 10 mg/kg, about 0.1 mg/kg to about 5 mg/kg, about 0.1 mg/kg to about 2 mg/kg, about 0.1 mg/kg to about 1 mg/kg, about 0.1 mg/kg to about 0.5 mg/kg, about 0.5 mg/kg to about 100 mg/kg, about 0.5 mg/kg to about 75 mg/kg, about 0.5 mg/kg to about 50 mg/kg, about 0.5 mg/kg to about 25 mg/kg, about 0.5 mg/kg to about 10 mg/kg, about 0.5 mg/kg to about 5 mg/kg, about 0.5 mg/kg to about 2 mg/kg, about 0.5 mg/kg to about 1 mg/kg, about 1 mg/kg to about 100 mg/kg, about 1 mg/kg to about 75 mg/kg, about 1 mg/kg to about 50 mg/kg, about 1 mg/kg to about 25 mg/kg, about 1 mg/kg to about 10 mg/kg, about 1 mg/kg to about 5 mg/kg, about 1 mg/kg to about 2 mg/kg, about 2 mg/kg to about 100 mg/kg, about 2 mg/kg to about 75 mg/kg, about 2 mg/kg to about 50 mg/kg, about 2 mg/kg to about 25 mg/kg, about 2 mg/kg to about 10 mg/kg, about 2 mg/kg to about 5 mg/kg, about 5 mg/kg to about 100 mg/kg, about 5 mg/kg to about 75 mg/kg, about 5 mg/kg to about 50 mg/kg, about 5 mg/kg to about 25 mg/kg, about 5 mg/kg to about 10 mg/kg, about 10 mg/kg to about 100 mg/kg, about 10 mg/kg to about 75 mg/kg, about 10 mg/kg to about 50 mg/kg, about 10 mg/kg to about 25 mg/kg, about 25 mg/kg to about 100 mg/kg, about 25 mg/kg to about 75 mg/kg, about 25 mg/kg to about 50 mg/kg, about 50 mg/kg to about 100 mg/kg, about 50 mg/kg to about 75 mg/kg, or about 75 mg/kg to about 100 mg/kg). In some embodiments, an effective amount of a compound described herein is about 0.1 mg/kg, about 0.5 mg/kg, about 1 mg/kg, about 2 mg/kg, about 5 mg/kg, about 10 mg/kg, about 25 mg/kg, about 75 mg/kg, or about 100 mg/kg.

Compositions and formulations described herein can conveniently be presented in a unit dosage form. A unit dosage form refers to physically discrete units suitable as unitary dosages for human subjects and other patients, each unit containing a predetermined quantity of active material (e.g., a galloylated polyphenol provided herein, or a pharmaceutically acceptable salt or solvate thereof) calculated to produce the desired therapeutic effect, in association with a suitable pharmaceutical excipient. For example, compositions and formulations described herein can be formulated as, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

A dosage of a compound provided herein can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

In some embodiments, the pharmaceutical compositions provided herein can be formulated to improve bioavailability of a compound provided herein. For example, consumables (milk, ascorbic acid, black pepper), special preparations (liposomes, emulsions, nanoparticles), or modifications (acetylation, methylation) have been shown to substantially increase bioavailability of compounds such as galloylated polyphenols. See, e.g., Aatif M. Biomedicines. 11:2078, 2023; Mao T, et al. Foods. 13:515, 2024; and Lewandowska U, et al. J. Agric. Food Chem. 61:12183-99; 2013.

The pharmaceutical compositions provided herein include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, mucosal, oral, parenteral, percutaneous, peridural, rectal, respiratory (inhalation), subcutaneous, sublingual, submucosal, topical, transdermal, transmucosal, transtracheal, ureteral, urethral and vaginal.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound provided herein with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition includes any of the compounds provided herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

### EXAMPLES

### Example 1. Galloylated Polyphenols as Antithrombotic Antagonists of Thiol Isomerases

Protein disulfide isomerase (PDI) family thiol isomerases are multidomain oxidoreductases that function in thrombus formation. PDI antagonism or targeted genetic deletion blocks thrombus formation in several murine models of thrombosis. PDI has a domain structure of a-b-b′-a′ in which the a and a′ domains contain the Cys-Gly-His-Cys catalytic motifs responsible for disulfide shuffling and the b and b′ domains are the substrate binding domains.

Flavonoid quercetins and bepristats were identified as two structure independent classes of PDI inhibitors that act by binding the hydrophobic pocket in the b′ domain and eliciting allosteric modifications that impair oxidoreductase activity. While screening a flavonoid library for inhibitors of the SARS-CoV-2 main protease (M^{pro}), a M^{pro} inhibitor termed PGHG that blocked PDI reductase activity with an IC₅₀ of 1.5±0.6 μM was identified. When tested against isolated domains of PDI, PGHG inhibited a and a′ fragments as well as ab, b′xa′ and abb′x fragments, indicating that it acts on the a and a′ domains of PDI. Supportive of an activity at the a and/or a′ domains, PGHG inhibited other vascular thiol isomerases including ERp5, ERp46, ERp57, and ERp72 with IC₅₀s of 1.5, 3.9, 3.3, and 7.8 μM, respectively.

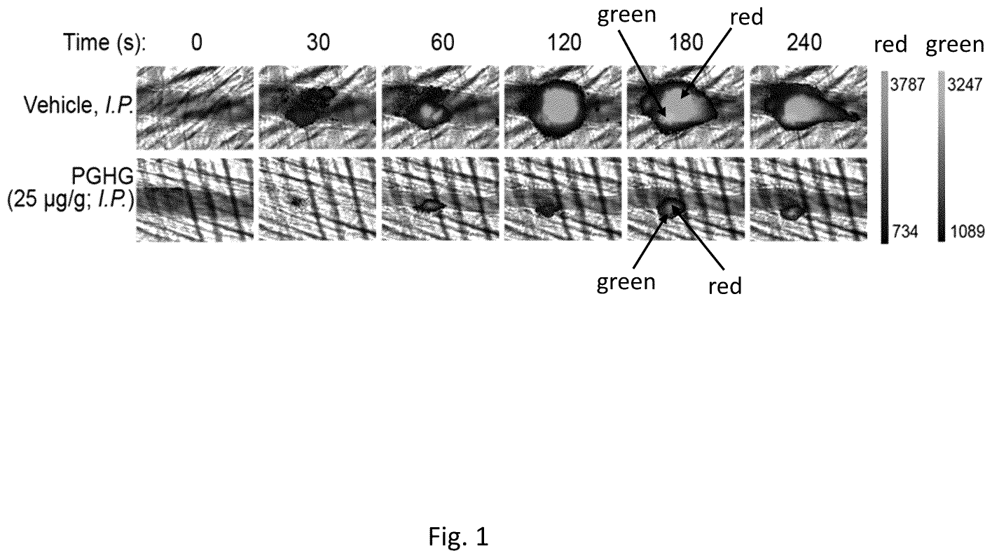

When tested in a cremaster muscle murine model of thrombus formation, PGHG (25 mg/kg) inhibited platelet accumulation to 18±6% of control levels and fibrin formation to 21±3% of controls (FIG. 1). In contrast, when tested in the mice tail transection model of bleeding, PGHG did not prolong the time to cessation of bleeding nor the total amount of blood loss. These results show that PGHG can inhibit thrombus formation without promoting bleeding.

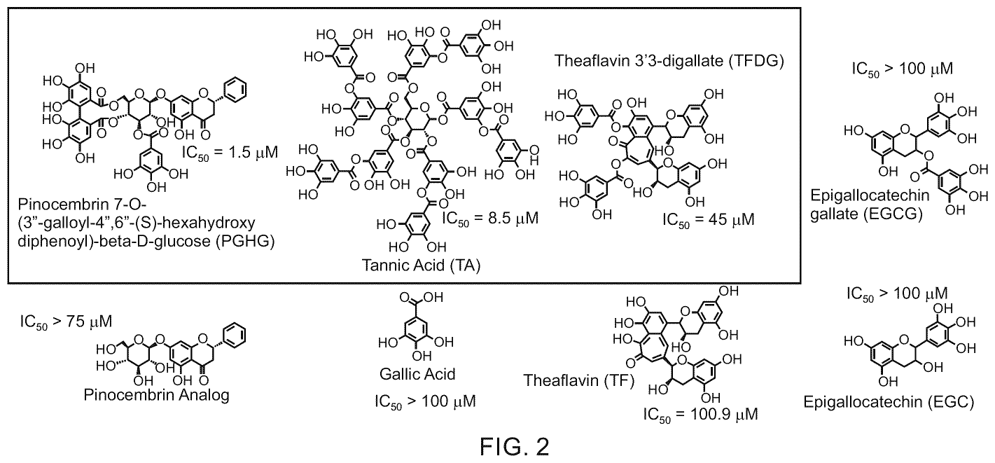

To better understand the mechanism by which PGHG inhibits PDI, other galloylated polyphenols were evaluated for their ability to inhibit PDI (FIG. 2). These studies identified galloylated polyphenols with structurally unrelated backbones, including theaflavin 3-3′-digallate (TFDG) and tannic acid (TA), that inhibited PDI. In contrast, related galloylated analogs such as epigallocatechin gallate (EGCG), and epicatechin-3-gallate (EGC) did not inhibit PDI. Similarly, the isolated backbones of active compounds, including pinocembrin (backbone of PGHG), theaflavin (backbone of TFDG), and gallate (backbone of TA) failed to inhibit PDI. Like PGHG, TFDG inhibited isolated a and a′ fragments as well as ab, b′xa′ and abb′x fragments, indicating that it, like PGHG, acts on the a and a′ domains of PDI. These structure-activity relationships show that a subgroup of galloylated polyphenols can inhibit PDI via interactions with the a and/or a′ domains.

Thrombosis is a common complication of COVID19. Our evaluation of plasma PDI levels in patients with mild (n=16), moderate (n=21), and severe (n=23) COVID19 showed that PDI levels increase according to the level of disease severity. Since previous studies have identified galloylated polyphenols that inhibit SARS-CoV-2 replication in vitro and in vivo (Furukawa, Sci Rep, 2021), PGHG was evaluated for its ability to inhibit the proteolytic activity of SARS-CoV-2 M^{pro} and to block SARS-CoV-2 in a Vero E6-based viral replication assay. PGHG inhibited M^{pro} with an IC₅₀ of 6.5 μM and SARS-CoV-2 replication with an IC₅₀ of 4.9 μM. To better understand the interaction of PGHG with M^{pro}, we performed all-atom, explicit water molecular dynamic simulations. These simulations showed an overall binding energy (ΔGₜₒₜₐₗ) of −58.64+3.17 and demonstrated hydrogen bonding between the galloyl group of PGHG and M^{pro} Cys145, the active site cysteine of the protease. Similarly, published molecular dynamic simulations of TA with M^{pro} show hydrogen bonding of TA with M^{pro} Cys145 (Haddad et al., Int. J. Mol. Sci., 2022). These observations suggest a mechanism wherein steric interactions of select galloylated polyphenols position a galloyl group for interactions with active site cysteines, as occur in both PDI and M^{pro}, inhibiting their activity.

These studies thus identify a group of orally available galloylated polyphenols that modify vascular thiol isomerases, inhibit thrombus formation, and block SARS-CoV-2 replication. Galloyltransferases are found in many plants and galloylated polyphenols are abundant in commonly consumed beverages such as teas. Members of this class of compounds may be useful in the setting of COVID19 as inhibitors of thrombosis and viral replication.

### Example 2. Galloylated Polyphenols as Antithrombotic Agents with Broad Activity Against Thiol Isomerases

Gallic acid is capable of free radical scavenging and galloylated polyphenols evolved, in part, to protect plants from oxidative stress (Karas D, et al. Food and Chemical Toxicology. 105:223-40, 2017; Ahmad M Z, et al. Frontiers in Plant Science. 11:848, 2020) and viral infection (Kumar S, et al. In: Lone R, Shuab R, Kamili A N, eds. Plant Phenolics in Sustainable Agriculture: Volume 1. Singapore: Springer Singapore, 2020, 517-32). Mammals lack the galloyltransferase required to synthesize galloylated polyphenols, so ingestion from plant sources represents the only means to acquire these compounds. Galloylated polyphenols are enriched in commonly consumed beverages such as tea and coffee and in high concentrations in dietary supplements. Although not uniformly positive, large epidemiologic studies evaluating the cardiovascular effects of tea and coffee consumption have generally indicated improved overall survival and decreased cardiovascular disease with tea and coffee consumption. See, e.g., Kuriyama S, et al. JAMA. 296:1255-65, 2006; Freedman N D, et al. NEJM. 366:1891-904, 2012. Antithrombotic effects have been noted among several potential cardiovascular protective activities. However, the identification of active constituents and the underlying mechanism of the antithrombotic activity of galloylated polyphenols action is largely unknown.

In order to identify compounds that inhibit both SARS-CoV-2 replication and thrombosis, a library of 1019 diverse flavonoids was screened for the flavonoids' ability to inhibit SARS-CoV-2 main protease (M^{pro}). See Lin L, et al. iScience. 26:107602, 2023. A galloylated flavonone termed pinocembrin 7-O-(3″-galloyl-4″,6″-(S)-hexahydroxydiphenoyl)-beta-D-glucose (PGHG) inhibited both M^{pro} and SARS-CoV-2 viral replication.

This study presents evidence that PGHG also inhibits thiol isomerases including PDI, ERp57, ERp5, ERp72, and ERp46 and is antithrombotic in vivo. These studies thus identify a large group of commonly consumed galloylated polyphenols that inhibit prothrombotic PDI family thiol isomerases through interactions with their catalytic domains.

### Methods

Recombinant protein purification. Recombinant ‘double-tagged’ (streptavidin-binding protein-tagged and FLAG-tagged) full-length PDI, ERp57, recombinant His-tagged full-length ERp5, ERp72, PDI domain fragments, and PDI mutant were cloned into a pET-15b vector at the NdeI and BamHI sites and transformed into Escherichia coli Origami B (DE3) cells (EMD Chemicals). The recombinant proteins were expressed and isolated by affinity chromatography with complete His-Tag purification resin (Roche Applied Science) or Pierce High-Capacity Streptavidin Agarose beads and purified on a Superdex 200 (GE Healthcare).

Di-eosin-GSSG disulfide reductase assay. The probe di-eosin glutathione disulfide, di-eosin-GSSG, was prepared by incubating 100 μM of GSSG with 1 mM of eosin isothiocyanate in 100 mM potassium phosphate containing 2 mM EDTA (pH 7.4), at 25° C. overnight in the dark. The mixture was passed down a PD-10 column and different fractions were collected. A fold change of fluorescence (Ex: 520 nm, Em: 550 nm) was calculated using samples subjected to either vehicle or 20 mM of DTT. Any fraction with a fold change >5 was kept. Using a stock of 10 mM eosin isothiocyanate, a standard curve was generated and concentration of samples were determined accordingly. Di-eosin-GSSG cleavage of purified thiol isomerases and PDI domains was monitored in a 96-well fluorescence plate format. PDI, CGXC-PDI variants, ERp5, ERp57, ERp46 and PDI domains were assayed at 50 nM in the presence or absence of the indicated small molecules. The assay included a thiol isomerase assay buffer (39 mM KH₂PO₄, 61 mM K₂HPO₄, 2 mM EDTA, 5 M DTT, pH 7.4) and 150 nM of the di-eosin-GSSG probe. The increase in fluorescence was determined for 60 min by excitation at 520 nm and emission at 550 nm in a Synergy Biotek 4. The reduction of 150 nM di-eosin-GSSG by 5 μM DTT in the presence or absence of mentioned small molecule or proteins served as a negative control.

To test the reversibility of the compounds, 20 μM PDI was incubated with 50 μM N-ethylmaleimide or PGHG for 30 min in thiol isomerase assay buffer. After 30 minutes of equilibration, the PDI-inhibitor mixture was diluted 100-fold with thiol isomerase assay buffer where the final concentration of enzyme is 0.2 μM and compounds at 500 nM or 50 μM. Di-eosin-GSSG was added to a final concentration of 300 nM and the assay was immediately recorded for 1 h.

Insulin reductase assay. Insulin was prepared by mixing 12.5 mg/mL insulin in 50 mM Tris-HCl, pH 8. 1 M HCl was carefully added to drop the pH to 2.0 for solution clarification. Once the solution clarifies, 1 M NaOH was added to rapidly adjust the pH back to 8.0. The solution was then adjusted to 1.5 mM (10 mg/mL) with 50 mM Tris-HCl, aliquoted and stored in −20° C. until use. 2× concentrations of thiol isomerases were prepared (final concentrations as indicated in each assay) and mixed with 1.2 mM DTT (final 0.3 mM) and 0.4 mM insulin (final 0.1 mM) using a thiol isomerase activity assay buffer. Insulin precipitation from enzyme-mediated reduction was assayed in 96-well clear bottom plates at 650 nm absorption.

Maleimide-pegylated Biotin (MPB) binding. MPB-binding experiments were performed in a reaction volume of 37.5 μL of polyphenols with 5 μM of thiol isomerase in phosphate buffered saline (PBS). The reaction mixture was incubated at 37° C. for 1 h. Subsequently, the reaction mixtures were incubated with 25 μM of MPB. The labelling was performed for 20 min at 25° C. A total of 12.5 μL of 4× Laemmli Sample Buffer with 10% v/v β-mercaptoethanol was added to each of the samples, followed by heating at 95° C. for 10 min. From each sample, 10 μL was loaded on a 4-15% SDS-PAGE TGX gel, followed by transfer on a nitrocellulose membrane. After blocking the membrane with tris-buffered saline containing 0.1% v/v Tween-20 (TBS-T) containing 5% BSA for 1 h, the membrane was incubated with a 1:2,000 dilution of streptavidin-HRP in TBS-T for an hour in the dark. Detection of the immunoblots was performed using a BioRad ChemiDoc MP imager.

Laser-injury thrombosis model. Thrombus formation in response to laser injury was measured as described previously. See Grover S P, et al. J. Thromb. Haemost. 18:3078-85, 2020; Koseoglu S, et al. Arterioscler. Thromb. Vasc. Biol. 33:481-8, 2013; and Matsuura S, et al. Arterioscler. Thromb. Vasc. Biol. 40:e262-e72, 2020. Briefly, cremaster muscle arterioles were injured using a MicroPoint Laser System (Andor, Belfast, UK). Platelet and fibrin accumulation were measured by infusion of Dylight 649-labeled anti-platelet (CD42b; 0.1 μg/g body weight; Emfret Analytics) and DyLight 488-labeled anti-fibrin (clone 59D8; 0.3 μg/g) antibodies through a jugular vein catheter. Data were acquired before and after laser injury using the brightfield, 488/520 nm, and 640/670 nm channels. Images were captured for 250 seconds at 2 frames/s using a CCD camera (ORCA Flash 4.0, Hamamatsu Photonics, Japan) on an AX-70 fluorescence microscope (Olympus, Japan). Data were analyzed using Slidebook 6.0 (Intelligent Imaging Innovations). Data from 16 to 30 thrombi were used to determine the median value of the integrated fluorescence intensity to account for variability in thrombus formation under any given experimental condition. AUC was calculated for individual thrombi and normalized to injury lengths to evaluate statistical significance. Injury lengths were determined as previously described in Grover S P, et al. J. Thromb. Haemost. 18:3078-85, 2020.

Ferric chloride thrombosis model. Thrombus formation induced by ferric chloride was assayed as previously described in Li W, et al. Circ. Res. 115:997-1006, 2014. The mice were treated by oral gavage with 100 μg/g punicalagin or vehicle control for 90 min prior to injury. Before the 90 min time point, the mice were anesthetized with a cocktail of 125 mg/kg ketamine and 12 mg/kg xylazine. The left jugular vein was cannulated for infusion of a DyLight 649 conjugated antibody against GPIbβ (Emfret Analytics; 0.1 μg/g) and 50 mg/kg pentobarbital for maintenance anesthetics. The right carotid artery was isolated with a piece of non-fluorescent plastic placed under the vessel. Vessel autofluorescence (excitation wavelength 488 nm, 50 ms) and platelet accumulation (excitation 660 nm, 50 ms) were captured for 15 sec (2 frame/sec) every min up to 30 min. The injury was made by topical application of a 1×2 mm filter paper saturated with 10% FeCl3 for 3 min. Thrombi formation was observed using a 10×0.3 NA water immersion objective mounted to an AX-70 fluorescence microscope (Olympus Japan) equipped with a CCD camera (ORCA Flash 4.0, Hamamatsu Photonics, Japan). Data were analyzed by Slidebook 6.0 (Intelligent Imaging Innovations).

Tail clip assay of hemostasis. Wildtype C57Bl/6J mice were anesthetized with 2.5% isoflurane or with a cocktail of ketamine and xylazine (125 mg/kg and 12 mg/kg, respectively) and maintained on a heat pad. The tails were transected at about 5 mm from the tip (1 mm diameter) prior to immersing into a 37° C. saline solution. The total bleeding time was recorded for 15 min. Red blood cells were then pelleted by centrifugation at 300×g for 6 min. The pellet was then lysed with 1 mL of red blood cell lysis buffer. Hemoglobin concentration was measured against a standard curve of purified hemoglobin at 575 nm absorbance.

Molecular docking. Smina (v2021-08-23) was employed to examine the interaction between PGHG and PDI in the context of potential conformation and orientation of the ligand at the binding site. The crystal structure of PDI (PDB: 4EL1 or 4EKZ) served as the receptor. PGHG was docked to the a domain of PDI (ligand_autobox). Subsequently, a conformation search algorithm was executed to investigate the various conformational states of the flexible ligand. Grid maps were utilized to evaluate the ligand-protein interaction at each point during the docking simulation. The docking results were clustered to detect similar conformations and to identify the conformation with the lowest binding free energy.

Statistics. The data were analyzed by one-way ANOVA with Dunnett's posthoc analysis or with two sample t test unless otherwise indicated. A P-value of <0.05 was considered statistically significant. Data were represented as mean±SEM unless otherwise indicated.

### Results

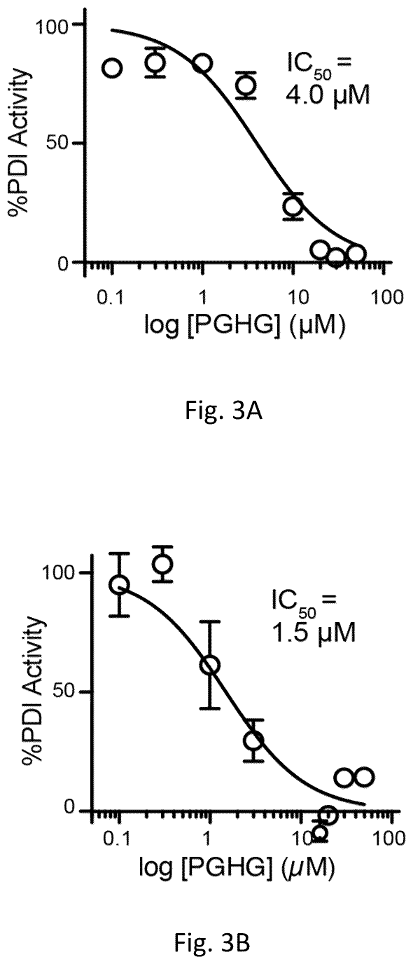

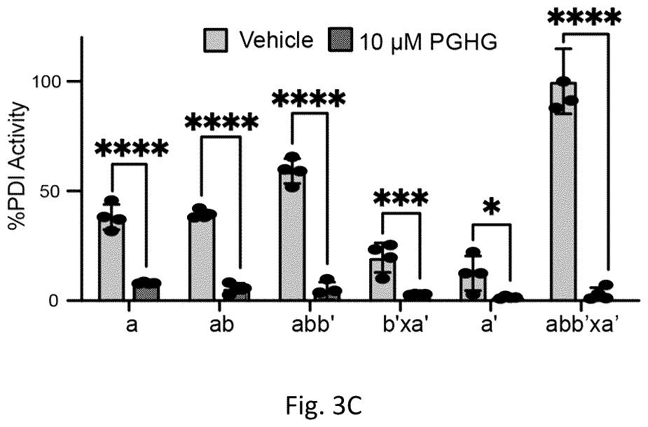

PGHG inhibits PDI by interacting with the catalytic domain. Pinocembrin 7-O-(3″-galloyl-4″,6″-(S)-hexahydroxydiphenoyl)-beta-D-glucose (PGHG) and apigenin were identified as inhibitors of SARS-CoV-2. See Lin L, et al. iScience. 26:107602, 2023. To identify flavonoids that inhibit both thrombus formation and SARS-CoV-2 infection, PGHG was tested for its ability to inhibit PDI. PGHG inhibited PDI in the low micromolar range in both insulin reductase and a di-eosin-GSSG reductase assays (FIGS. 3A and 3B; see also Example 1). To assess the mechanism of PDI inhibition by PGHG, the ability of PGHG to inhibit the reductase activity of separate domains of PDI was evaluated. PGHG inhibited activity of all fragments including the isolated the a and a′ domains, indicating that it acts at both the catalytic domains of PDI (FIG. 3C; see also Example 1).

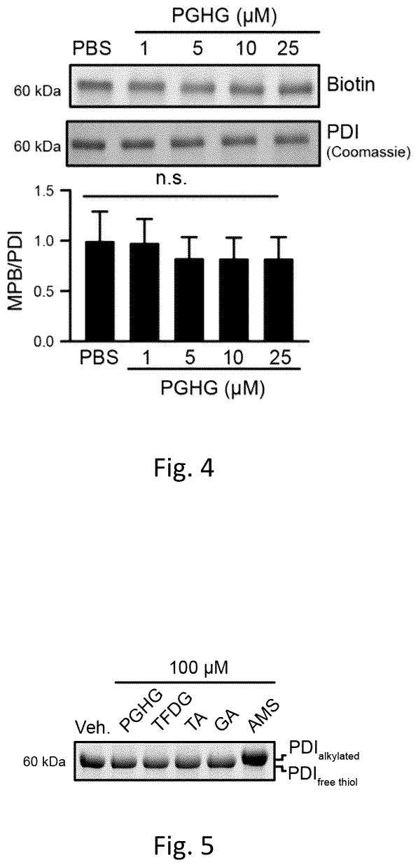

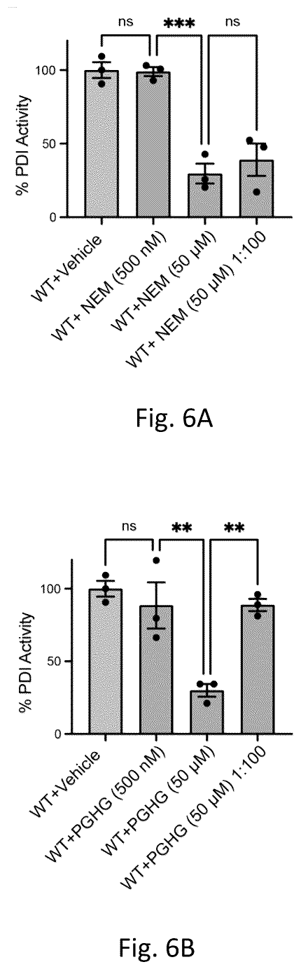

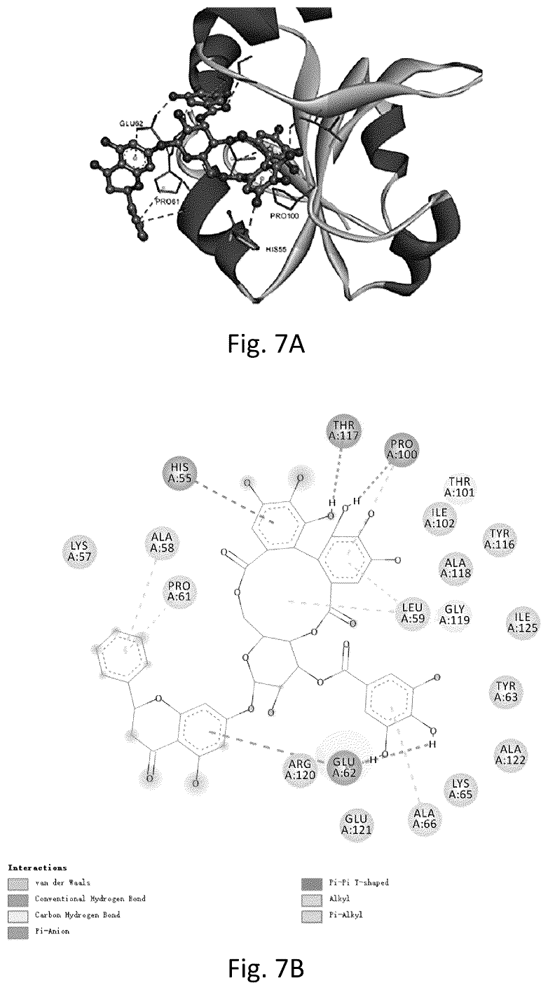

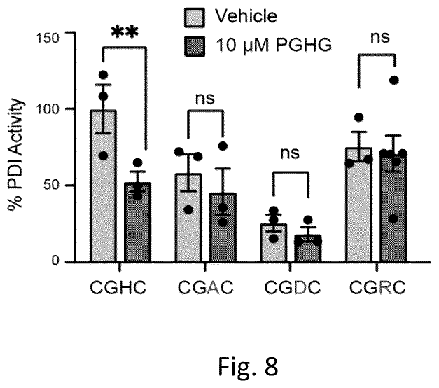

Inhibition of the catalytic domains of PDI by PGHG could occur via a covalent interaction involving the active site cysteines or by a reversible mechanism. To assess whether PGHG acts at the catalytic cysteines, it was determined whether PGHG interferes with the binding of maleimide-pegylated biotin (MPB), which interacts with free thiols. Preincubation with PGHG had no significant effect on MPB binding (FIG. 4). Covalent interaction was also tested by potential alkylation of PGHG to the protein, which may cause an apparent higher molecular weight shift in a sodium dodecyl sulfate polyacrylamide gel. PGHG did not induce a shift in molecular weight (FIG. 5). 4-acetomido-4′-maleimidylstilbene-2,2′ disulfonic acid (AMS), which shifts the mass of the protein by 500 Da for every cysteine alkylated, was used as a positive control. AMS induced a slight increase in molecular mass indicating alkylation of the free cysteines. To assess whether or not binding of PGHG was reversible, jump dilution experiments were performed. N-ethylmaleimide (NEM) was used for a control experiment because covalent alkylation of the free thiols will expectedly prevent PDI activity (FIG. 6A). Consistent with the observation that PGHG did not interfere with MPB binding, PGHG inhibitory activity was lost following a 100-fold dilution, indicating that it is a reversible inhibitor (FIG. 6B). Molecular docking of the binding of PGHG to the a domain of PDI demonstrated an interaction of a galloyl moiety with His55 within the Cys53-Gly54-His55-Cys56 catalytic motif (FIGS. 7A and 7B). PDI mutants in which His55 and His399 were mutated to Ala, Asp, or Arg showed variable levels of reductase activity compared to WT PDI. However, these mutants were resistance to PGHG (FIG. 8), underscoring the importance of His55 and His399 in PGHG inhibitory activity.

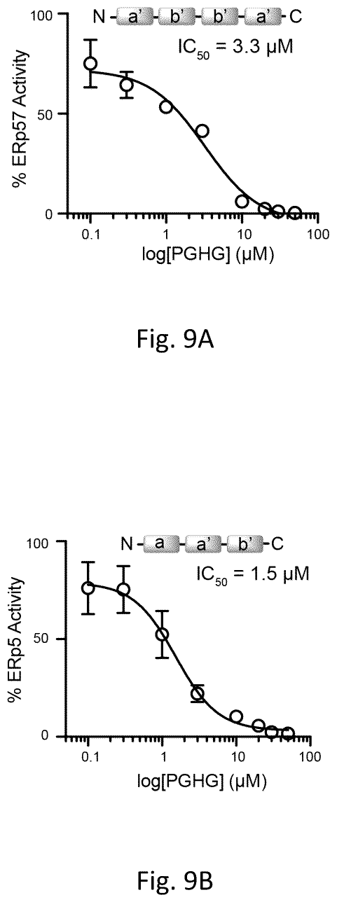

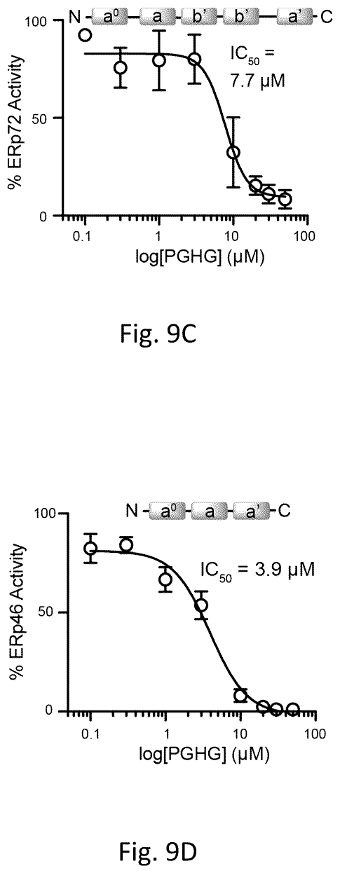

PGHG inhibits multiple thiol isomerases. Thrombus formation is promoted by several vascular thiol isomerases in addition to PDI. These thiol isomerases, including ERp57, ERp5, ERp72, and ERp46, possess at least one thioredoxin fold with a CGHC catalytic motif. The inhibition of these thiol isomerases by PGHG was evaluated. Each of the vascular thiol isomerases was inhibited by PGHG with relatively similar IC₅₀s: ERp57 (3.3 μM), ERp5 (1.5 μM), ERp72 (7.8 μM), and ERp46 (3.9 μM) (FIGS. 9A-9D). The fact that these isomerases demonstrate more homology in their catalytic (a, a′) domains compared to their binding domains (b, b′) provides further support that the compound acts at the catalytic domains of thiol isomerases.

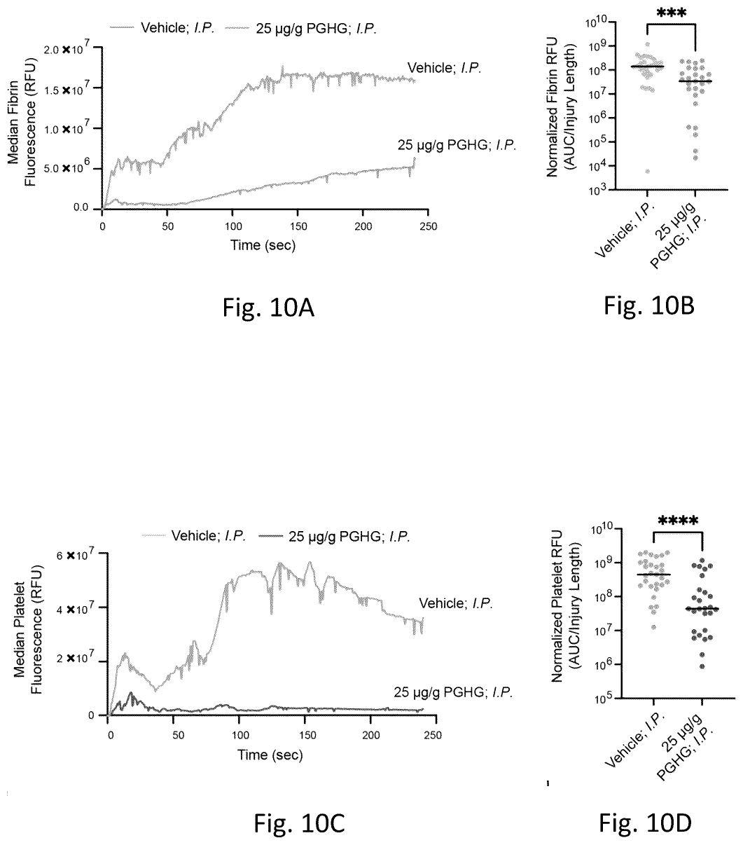

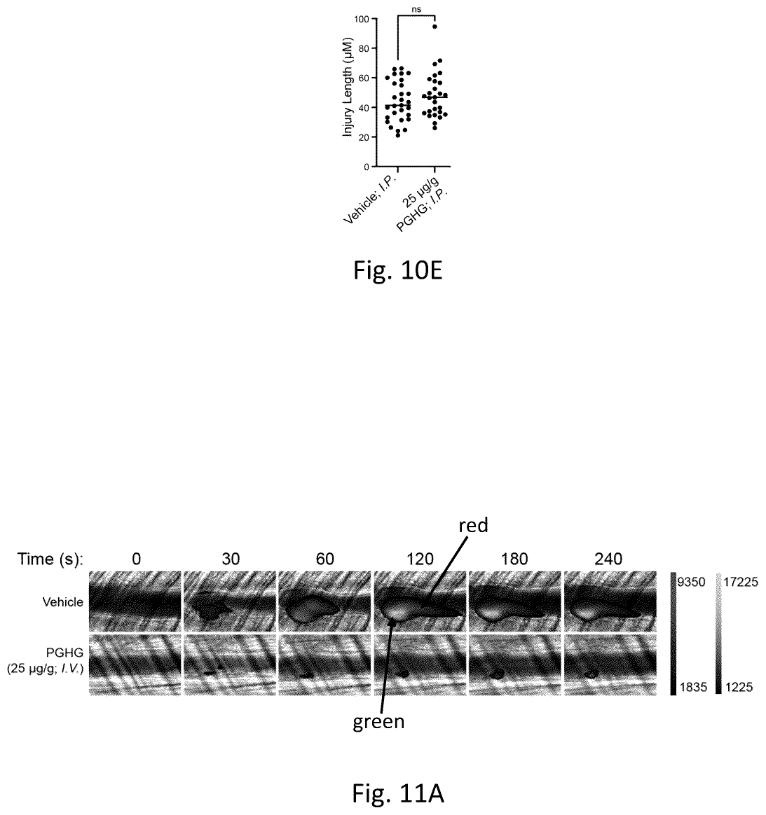

Inhibition of thrombus formation by PGHG. Previously identified inhibitors of PDI including small molecules, antibodies, and peptides have been shown to block thrombus formation in vivo. As described in Example 1, to determine whether PGHG inhibits thrombus formation, mice were exposed to 25 μg/g PGHG via intraperitoneal injection and the effects on platelet accumulation and fibrin formation that occur following laser injury of cremaster arterioles were evaluated (FIG. 1). PGHG injection resulted in a decrease in mean fibrin fluorescence to 33.44±7.53% of control (FIGS. 10A and 10B) and a decrease in mean platelet accumulation to 29.56±9.08% of control (FIGS. 10C and 10D). The differences in platelet accumulation and fibrin formation were not secondary to differences in the extent of laser injury since injury size between control and PGHG-treated mice was indistinguishable (FIG. 10E).

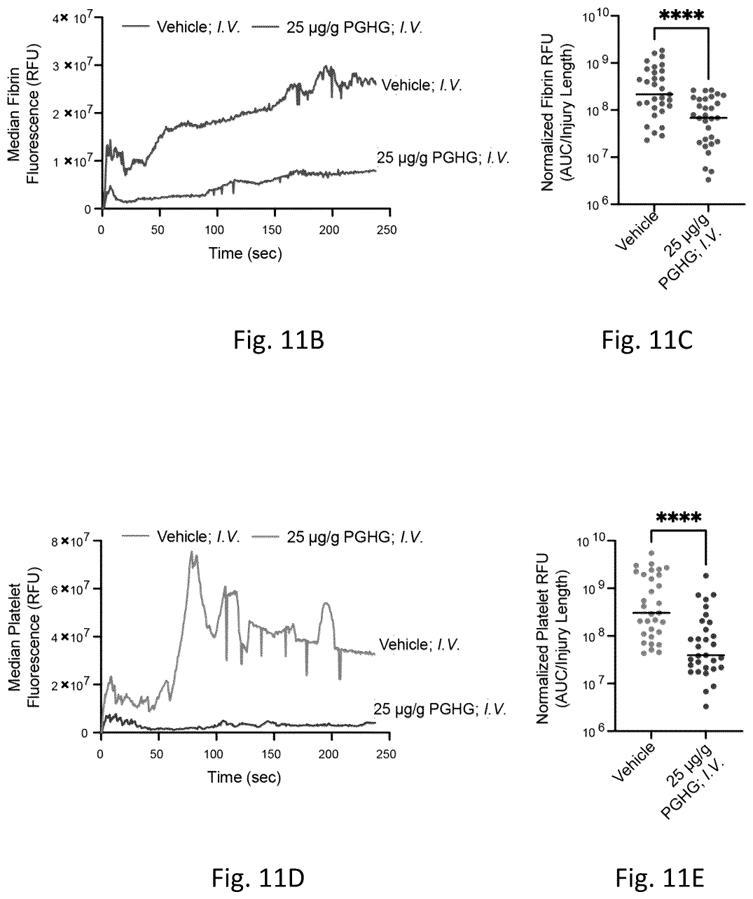

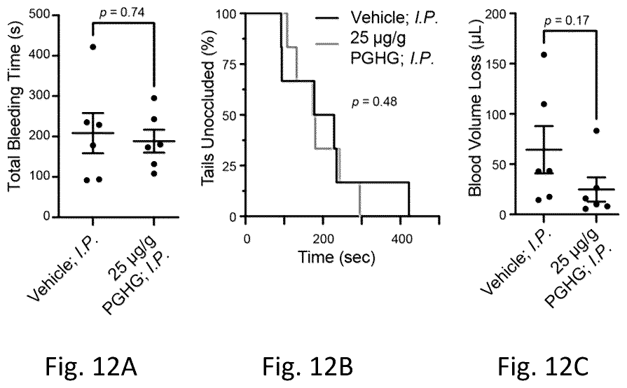

Intravenous infusion of PGHG also inhibited both fibrin formation and platelet accumulation following laser injury of cremaster arterioles (FIGS. 11A-E), showing 21±3.7% and 18±6.2% fluorescence compared to controls, respectively. In contrast to its effect on thrombus formation, PGHG injection did not prolong the time required for bleeding to subside following tail snip (FIGS. 12A and 12B), nor did it significantly affect total blood loss in this assay (FIG. 12C).

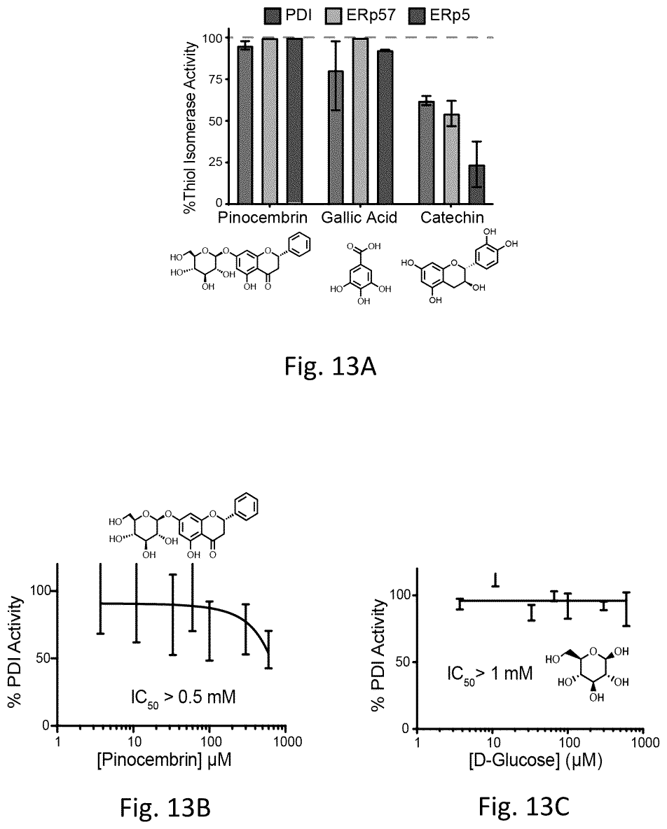

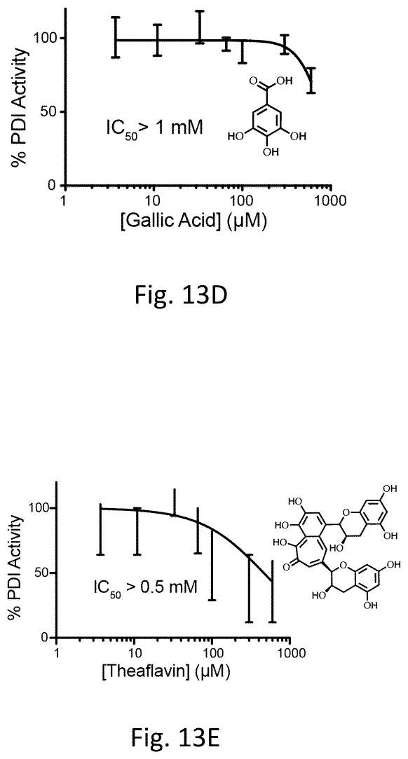

Galloylated polyphenols inhibit thiol isomerases. As PGHG inhibits thiol isomerases and thrombus formation in vivo, which moieties within PGHG are active was evaluated. PGHG is a modification of pinocembrin that is both glycosylated and galloylated. Neither pinocembrin, gallic acid nor glucose showed substantial activity against thiol isomerases (FIGS. 13A-E). The observation that pinocembrin alone had no activity prompted the evaluation of the role of polyphenol galloylation in thiol isomerase antagonism.

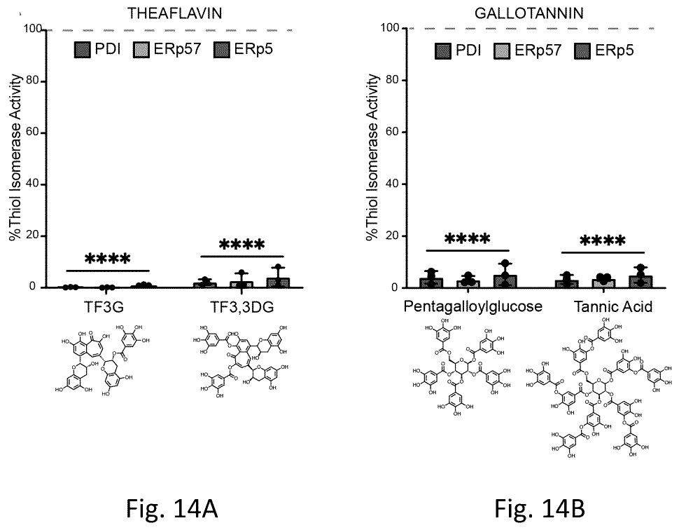

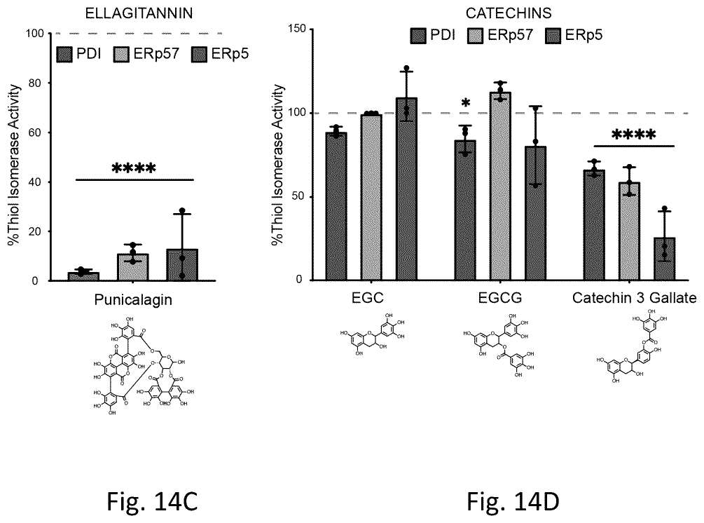

Galloylation is a common modification of plant polyphenols, and galloylated polyphenols are highly abundant in commonly consumed ingestibles such as tea, coffee, fruits and vegetables. Several families of galloylated polyphenols were tested for their ability to inhibit thiol isomerases. Specifically, galloylated polyphenols were tested against recombinant PDI, ERp57, and ERp5 at 20 μM compound in a di-eosin-GSSG assay (FIGS. 14A-D). The galloylated theaflavins, theaflavin-3-gallate and theaflavin-3,3-gallate, showed strong inhibition of all three thiol isomerases (FIG. 14A). Non-galloylated theaflavin demonstrated poor inhibition (FIGS. 13A-E). Similarly, gallotannins including pentagalloylglucose and tannic acid potently inhibited all thiol isomerases tested, while glucose did not (FIG. 14B; FIGS. 13A-E). Punicalagin also showed strong inhibition of thiol isomerases (FIG. 14C). Epigallocatechin (EGC) and epigallocatechin gallate (EGCG) showed relatively little activity (FIG. 14D), whereas catechin-3-gallate showed some inhibition, particularly of ERp5. The activity of catechin-3-gallate was similar to the non-galloylated analog, catechin (FIGS. 13A-E). Evaluation of the physicochemical parameters of compounds tested for their ability to inhibit thiol isomerase reductase activity in the di-eosin-GSSG assay demonstrated commonalities among inhibitory compounds. All polyphenols that potently inhibited (i.e., >50% inhibition at 20 μM) each thiol isomerase tested contained at least one galloyl moiety (FIGS. 14A-D). See also Table 1.

TABLE 1
Physiochemical properties and PDI inhibitory activity of tested dietary phenolics.

| | | | | | |
|---|---|---|---|---|---|
| | | MW | Polar Surface | Relative PDI | Rotatable |
| Compounds | cLogP | (Da) | Area (Å²) | inhibition | Bonds |

Theaflavins

| | | | | | | |
|---|---|---|---|---|---|---|
| Theaflavin | 0.6 | 564.5 | 218 | >500 | μM | 2 |
| Theaflavin 3-gallate | 3.5 | 716.6 | 284 | 3.4 | μM | 5 |
| Theaflavin 3,3′-gallate | 4.7 | 868.7 | 351 | 3.0 | μM | 8 |

Flavonoids

| | | | | | | |
|---|---|---|---|---|---|---|
| Pinocembrin | 2.7 | 256.25 | 66.8 | >500 | μM | 1 |
| PGHG | 3.6 | 872.14 | 346 | 1.5 | μM | 6 |

Gallotannins

| | | | | | | |
|---|---|---|---|---|---|---|
| Tannic acid | 6.2 | 1701.2 | 778 | 8.5 | μM | 31 |
| Pentagalloyl-glucose | 3.6 | 940.7 | 444 | 2.9 | μM | 16 |
| Gallic Acid | 0.7 | 170.1 | 98 | >500 | μM | 1 |

Punicalagins

| | | | | | | |
|---|---|---|---|---|---|---|
| Punicalagin | 1.7 | 1084.7 | 511 | 19 | μM | 0 |

Catechins

| | | | | | | |
|---|---|---|---|---|---|---|
| Catechin | 0.4 | 290.3 | 110 | >20 | μM | 1 |
| EGC | 1.5 | 442.4 | 177 | >500 | μM | 4 |
| EGCG | 1.2 | 458.4 | 197 | 243 | μM | 4 |
| Catechin 3-gallate | 1.5 | 442.4 | 177 | >20 | μM | 4 |

As a group, catechins did not potently inhibit PDI, although catechin and catechin-3-gallate had activity against ERp5 (FIGS. 13A-E; FIGS. 14A-D). Of the compounds tested, galloylated polyphenols that showed potent, pan-thiol isomerase inhibition were larger than less active compounds, with molecular weights greater than 700 Da and polar surface areas of >250 Å (Table 1). In contrast to size, hydrophobicity was not obviously correlated with thiol isomerase inhibition. In particular, punicalagin showed strong thiol isomerase inhibition, but is not strongly hydrophobic.

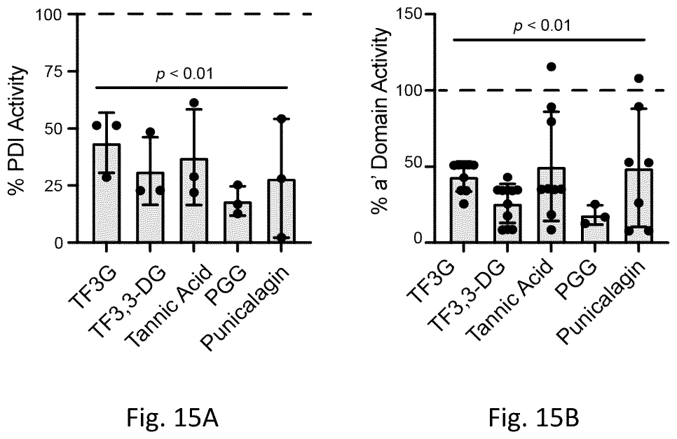

The ability of large, galloylated polyphenols to inhibit PDI reductase activity was further characterized. Since theaflavin-3-gallate, theaflavin-3,3-gallate, pentagalloylglucose, tannic acid, and punicalagin demonstrated inhibitory activity against multiple thiol isomerases that share a relatively conserved catalytic domain, the ability of these compounds to inhibit the isolated a′ domain of PDI was evaluated. Like PGHG, all these galloylated polyphenols inhibited the ability of isolated a′ domain to cleave di-eosin-GSSG (FIGS. 15A and 15B). The ability of these galloylated polyphenols to inhibit was also evaluated using the insulin reductase assay. Theaflavin-3-gallate, theaflavin-3,3-gallate, pentagalloylglucose, tannic acid, and punicalagin all inhibited the ability of PDI to reduce insulin (FIG. 15B). These studies thus identify a large group of galloylated polyphenols that inhibit PDI family thiol isomerases through interactions with their catalytic domains.

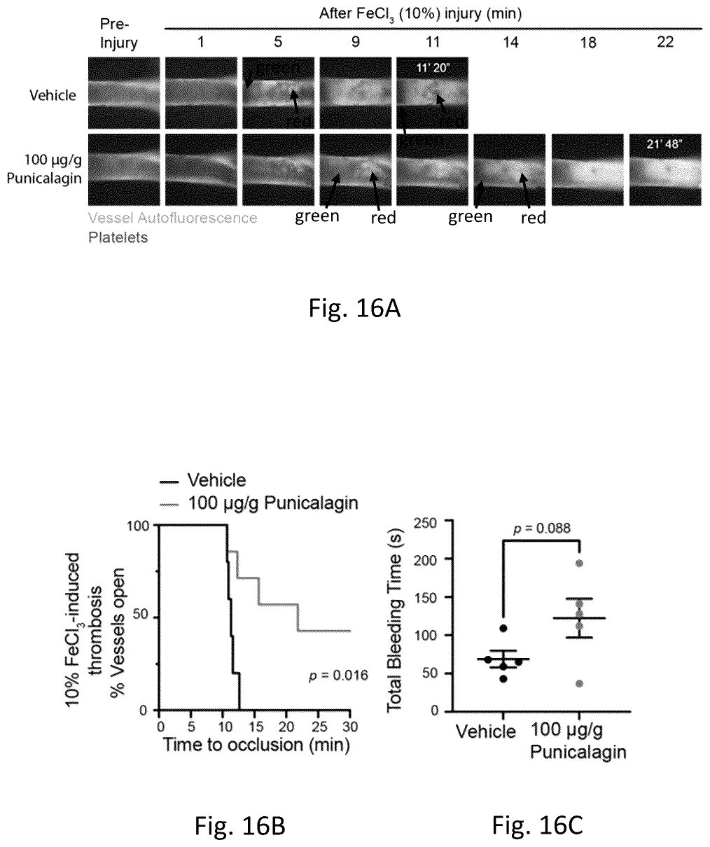

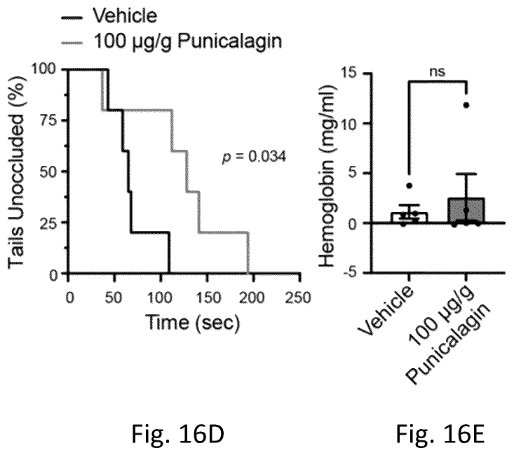

Punicalagin inhibits thrombus formation. Galloylated polyphenols are abundant in commercially available supplements. One common example is punicalagin in pomegranate extracts and supplements. The ability of orally delivered punicalagin to inhibit thrombus formation following vascular injury was assessed. For these studies, mice were treated with 100 mg/g punicalagin via oral gavage. A filter paper soaked in a solution of 10% FeCl₃ was applied to the carotid artery for 3 minutes after gavage. Vessel patency was visualized by fluorescence microscopy using anti-GPIbβ antibody to monitor for vascular occlusion. Under these conditions, 50% of mice showed occlusion at approximately 11 minutes and all by 13 minutes when gavage included only vehicle (FIGS. 16A and B). In contrast, in mice who received punicalagin by oral gavage, nearly 50% of vessels never occluded (FIG. 16B). The time for occlusion of vessels that clotted despite punicalagin ingestion was consistently prolonged compared to controls. In the 1 mm diameter tail transection model of hemostasis, there was a trend towards a prolonged total bleeding time to 110 sec in mice ingesting punicalagin compared to 70 sec for vehicle control mice (FIG. 16C). Plotting the data as Kaplan Meier curves indicated prolonged bleeding (FIG. 16D) despite similar levels of hemoglobin loss between the two groups (FIG. 16E), suggesting that punicalagin ingestion may potentially impact pro-hemostatic functions.

### Discussion

These studies show that galloylated polyphenols represent a large class of thiol isomerase inhibitors with antithrombotic potential. The vast majority of galloylated polyphenols are either galloylated theaflavins, gallotannins or pentagalloylglucose, ellagitannins, or galloylated catechins. By screening galloylated polyphenol analogs from different classes, it was shown that all galloylated polyphenols tested >700 Da inhibited thiol isomerases. The large inhibitory galloylated polyphenols include gallotannins and ellagitannins, galloylated theaflavins and galloylated flavonoids such as PGHG, thus demonstrating broad representation across most classes (Table 1). Based on these results, additional large galloylated polyphenols such as castalagin, vescalagin, and corilagin are predicted to inhibit thiol isomerase activity. Catechins showed some activity against thiol isomerases, particularly ERp5. However, for this class of polyphenols, the requirement for a galloyl group and large size are less clear. The identification of parameters that identify these antithrombotic compounds is important because of the large number of potential thiol isomerase inhibitors of galloylated polyphenol class and because of their dietary abundance.

Gallic acid is a redox reactive moiety incorporated by plants into polyphenols via galloyltransferases. The evidence that galloylated polyphenols act at the catalytic domain of diverse thiol isomerases raises the possibility that the galloyl group interacts covalently with active site cysteines within the thiol isomerases. However, evidence of covalent binding between galloylated polyphenols and thiol isomerases was not found (see FIG. 5 and FIG. 7B). Nonetheless, hydrogen bonding could occur between a hydroxyl of the galloyl group and the cysteine backbone (amide carbonyl oxygen of the cysteine backbone) or with other side chains in the vicinity of the CGHC catalytic motif.

The mechanism of action of galloylated polyphenols is distinct from that of quercetin flavonoids, which also inhibit PDI. Quercetin flavonoids interact with the b′ domain of PDI. NMR studies of PDI in association with quercetin-3-rutinoside showed that this flavonoid binds within the hydrophobic pocket of the b′ domain of PDI at His256. Structure-activity relationships using quercetin-3-rutinoside analogs coupled with molecular dynamic simulations showed that a phenoxyl group at position 7 within the flavonoid ring of quercetin-3-rutinoside interacts with His256 (see Liao X, et al. J. Agric. Food Chem. 70:4475-83, 2022). In contrast, the galloylated polyphenol, PGHG, acts at the catalytic domains (a and a′) of PDI (see FIGS. 3, 4, 6A, 6B, 8, and 9A-9D). This observation is consistent with the fact that PGHG inhibited other thiol isomerases with a CGHC motif within their catalytic domains, including ERp5, ERp57, ERp72, and ERp46. Molecular docking shows that PGHG orients in a pocket with the thioredoxin fold of the catalytic domains of PDI and interacts with histidine within the CGHC motif. Mutations of intervening sequences show that mutation of His55/His399 to Ala, Asp, or Arg reverses inhibition by PGHG, confirming a role for His55/His399. The lack of covalent interactions between PGHG and PDI is consistent with the observation that inhibition of PDI by PGHG is reversible. See FIGS. 4, 6A, 6B, 7A, and 8. Although we do not yet know whether or not all inhibitory galloylated polyphenols block thiol isomerases via a similar mechanism, our results indicate that galloylated polyphenols represent a class of compounds that impair the function of the active site motif within the thioredoxin-like catalytic domains.

The original screen of a 1019-compound flavonoid library was designed to identify compounds that both interfere with viral replication by blocking SARS-CoV-2 M^{pro} and are antithrombotic via inhibition of thiol isomerases. See Lin L, et al. iScience. 26:107602, 2023. M^{pro} is a cysteine-protease with a catalytic dyad including Cys145 and His41. From the compounds that inhibit M^{pro}, PGHG was selected to test as a PDI antagonist since it inhibited viral replication potently. See Lin L, et al. iScience. 26:107602, 2023. Molecular dynamic simulations indicated, among other interactions, hydrogen bond formation between a hydroxyl group of the galloyl moiety and His41 of the catalytic dyad. See Lin L, et al. iScience. 26:107602, 2023. Of note, the galloylated polyphenol theaflavin-3,3-digallate was another galloylated compound selected as one of the nine best M^{pro} inhibitors detected in the screen (FIGS. 14A-14D; Table 2). See also Lin L, et al. iScience. 26:107602, 2023. Other galloylated polyphenols that were identified as inhibitors of thiol isomerases also inhibit SARS-CoV-2 and M^{pro} (Table 2).

TABLE 2
Activity of active galloylated polyphenols against SARS-CoV-2 M^{pro}.

| | | | | Modeling shows gallic |
|---|---|---|---|---|
| | SARS-CoV-2 | | Inhibition of viral | acid interaction with |
| | M^{pro} inhibition | | replication | His41 or Glu166 |
| PGHG¹ | 6.5 | μM | Vero Cells | His41; Glu166 |
| | | | IC₅₀ − 5 μM | |
| Theaflavin 3-O- | 18.5 | μM | Vero Cells | His44; Glu166 |
| gallate² | | | | |
| Theaflavin 3,3′- | 9.5 μM; | | VeroE6/TMPRSS2 | His41; Glu166 |
| O-digallate³ | 45 μM | | Multiple SARS-CoV-2 | |
| | | | variants | |
| Punicalagin⁴ | 5.7 | μM | 293T-hACE2 | NA |
| | | | NIC-H460 | |
| | | | Multiple SARS-CoV-2 | |
| | | | variants | |
| Tannic acid⁵ | 3 μM | | 293T-hACE2 | His41; Glu166 |
| | 13.4 μM | | NIC-H460 | |
| | | | variants | |
| Pentagalloyl- | 25 | μM | Vero Cells | occludes His41; |
| glucose⁶ | | | IC₅₀ − 15 μM | reacts with Glu166 |

¹Lin L, et al. iScience. 26: 107602, 2023.
²Chauhan M, et al. Sci Rep. 12: 13146, 2022.
³Lin L, et al. iScience. 26: 107602, 2023; Yang F, et al. J. Integr. Med. 20: 488-96, 2022; Shin-Ya M, et al. Sci Rep. 13: 16577, 2023.
⁴Chen HF, et al. Elife. 12, 2023; Saadh MJ, et al. Eur. Rev. Med. Pharmacol. Sci. 25: 3908-13, 2021.
⁵Wang SC, et al. Am. J. Cancer Res. 10: 4538-46, 2020; Chen HF, et al. Elife. 12, 2023; Yang F, et al. J. Integr. Med. 20: 488-96, 2022; Haddad M, et al. Int. J. Mol. Sci. 23, 2022.
⁶Jin YH, et al. Biomedicines. 10, 2022; Chiou WC, et al. Biochem. Biophys. Res. Commun. 591: 130-6, 2022.

Tannic acid inhibits M^{pro} with a IC₅₀ of 13.4 μM and blocks SARS-CoV-2 virus infection in 293T cells expressing hACE2 (Wang S C, et al. Am. J. Cancer Res. 10:4538-46, 2020). Pentagalloylglucose also inhibits SARS-CoV-2 viral infection in culture and blocks viral cysteine-proteases (Jin Y H, et al. Biomedicines. 10, 2022). Punicalagin inhibits both SARS-CoV-2 M^{pro} and omicron variants of SARS-CoV-2 infection (Chen H F, et al. Elife. 12, 2023; Saadh M J, et al. Eur. Rev. Med. Pharmacol. Sci. 25:3908-13, 2021). Theaflavin 3-gallate inhibits M^{pro} with a IC₅₀ of 18.5 μM and reduces viral counts in a Vero cell assay by 75% (Chauhan M, Bhardwaj V K, Kumar A, et al. Sci. Rep. 12:13146, 2022). Molecular dynamic simulations consistently indicate that interactions of the galloyl moiety of these compounds with His41 and Glu166, which is a component of the oxyanion hole of the M^{pro} active site, are common. See Firouzi R, et al. Proteins. 90:1090-101, 2022; Moritsugu K, et al. J. Chem. Inf. Model. 63:240-50, 2023; and Table 2. Nonetheless, the possibility that the galloyl moiety of these galloylated polyphenols acts in the vicinity of the reactive cysteine in both M^{pro} and thiol isomerases has not been evaluated in high-resolution structural analyses and awaits future studies.

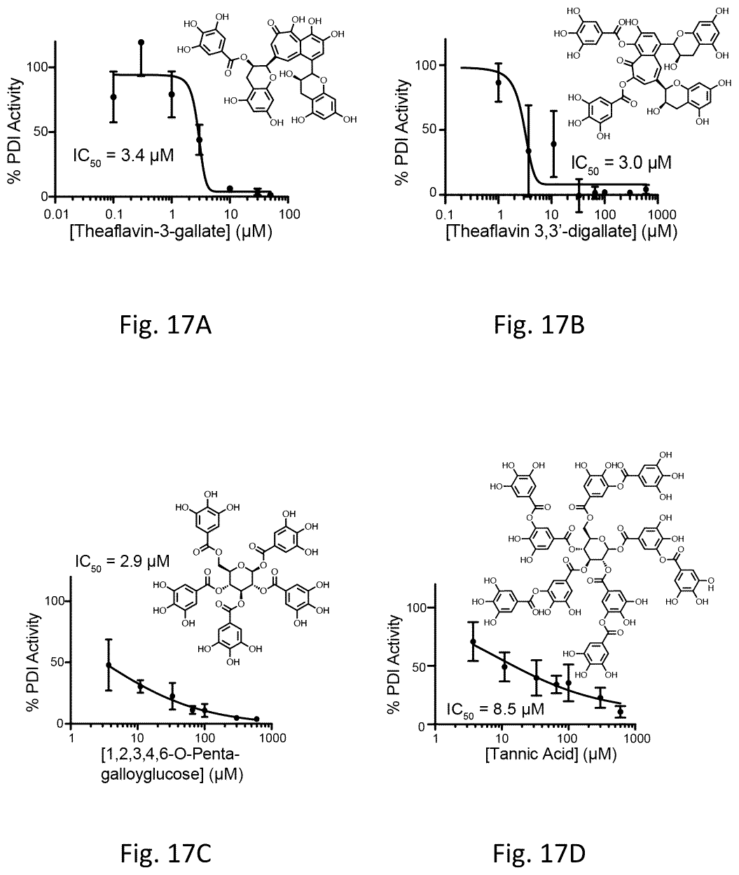

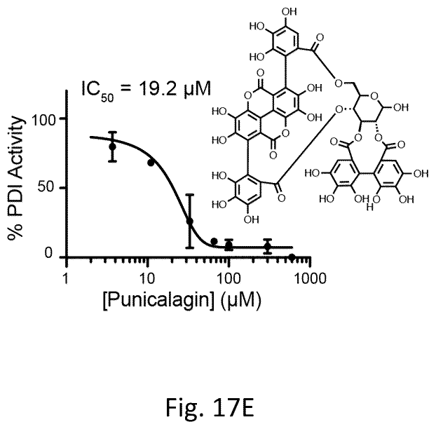

Although the finding that many commonly consumed galloylated polyphenols inhibit thiol isomerases (see FIGS. 14A-14D; FIGS. 17A-E) and several are antithrombotic (see FIG. 1; FIGS. 10A-10D; FIGS. 16A-E) has actionable implications for cardiovascular health, there are also limitations. Many galloylated polyphenols are poorly absorbed, have low bioavailability, and/or are extensively metabolized (Scalbert A, et al. Biomedicine & Pharmacotherapy. 56:276-82, 2002; Manach C, et al. Am. J. Clin. Nutr. 81:230s-42s, 2005). The plasma levels observed following ingestion of standard amounts of tea or other plant-derived foods and beverages are unlikely to be sufficient for inhibition of thiol isomerases. Nonetheless, ingestion of commercially available supplements results in circulating concentrations of galloylated polyphenols adequate to inhibit thiol isomerase activity and oral ingestion of punicalagin inhibits thrombus formation in vivo (FIGS. 16A-E). Methods to improve bioavailability using other consumables (milk, ascorbic acid, black pepper), special preparations (liposomes, emulsions, nanoparticles), or modifications (acetylation, methylation) have been shown to substantially increase bioavailability. See, e.g., Aatif M. Biomedicines. 11:2078, 2023; Mao T, et al. Foods. 13:515, 2024; and Lewandowska U, et al. J. Agric. Food Chem. 61:12183-99; 2013.

With regard to the antithrombotic mechanism of galloylated polyphenols, inhibition does not necessarily prove causation; however, without wishing to be bound by any theory, it is reasonable to believe that inhibition of thiol isomerase activity is associated with the ability to inhibit thrombus formation. These galloylated polyphenols have other potential antithrombotic targets in addition to thiol isomerases and there is no reason to expect that thiol isomerases would be the exclusive targets of these compounds. For example, tannic acid and 1,2,3,4,6-pentagalloylglucose (PGG) have antiplatelet activities. See, e.g., Ren L, et al. J. Cell Mol. Med. 24:14257-69; 2020; Marcińczyk N, et al. Front Pharmacol. 12:806891, 2021; Lee J J, et al. Food Chem. Toxicol. 69:94-101; 2014; Perveen R, et al. PLoS One. 6:e26238, 2011; Jeon W K, et al. J. Ethnopharmacol. 106:62-9; 2006. Nonetheless, thiol isomerases are both sensitive to these compounds and are well-recognized to function in thrombus formation.

Galloylated polyphenols represent a large class of antithrombotic compounds with broad activity against thiol isomerases. PGHG acts at the thiol isomerase catalytic domains. Structure activity relationships using a series of galloylated polyphenols demonstrate that several members of this class of compounds act at the catalytic domain of thiol isomerases. High molecular weight galloylated polyphenols including galloylated theaflavins, gallotannins, punicalagin, and galloylated flavonoids inhibited thiol isomerases. Oral administration of punicalagin inhibited thrombus formation in vivo.

Many of these compounds also inhibit SARS-CoV-2 M^{pro} and viral replication. The identification of a large subset of galloylated polyphenols capable of inhibiting thiol isomerases and SARS-CoV-2 M^{pro} raises the possibility that the galloyl group of these compounds can act in the vicinity of active site cysteines.

### Other Embodiments

A number of embodiments of the invention have been described. Nevertheless, it will be understood that various modifications may be made without departing from the spirit and scope of the invention. Accordingly, other embodiments are within the scope of the following claims.

1-52. (canceled)

53. A method of inhibiting a thiol isomerase in a subject in need thereof, the method comprising administering to the subject a therapeutically effective amount of a galloylated polyphenol, or a pharmaceutically acceptable salt thereof, wherein the galloylated polyphenol is selected from the group consisting of theaflavin 3-gallate, theaflavin 3,3′-digallate, pentagalloyl-glucose, gallic acid, punicalagin, EGCG, catechin 3-gallate, or a combination thereof.

54. The method of claim 53, wherein the galloylated polyphenol, or pharmaceutically acceptable salt thereof, inhibits one or more thiol isomerases selected from the group consisting of PDI, ERp46, ERp57, ERp5, and ERp72.

55. The method of claim 53, wherein the galloylated polyphenol, or pharmaceutically acceptable salt thereof, inhibits PDI, ERp57, and ERp5.

56. The method of claim 53, wherein the galloylated polyphenol, or pharmaceutically acceptable salt thereof, inhibits ERp5.

57. The method of claim 53, wherein the galloylated polyphenol, or pharmaceutically acceptable salt thereof, is administered at a dosage of about 0.1 mg/kg to about 100 mg/kg.

58. The method of claim 53, wherein the method comprises treating or preventing thrombosis.

59. The method of claim 58, wherein the galloylated polyphenol, or pharmaceutically acceptable salt thereof, inhibits fibrin formation in the subject.

60. The method of claim 58, wherein the galloylated polyphenol, or pharmaceutically acceptable salt thereof, inhibits platelet accumulation in the subject.

61. The method of claim 58, wherein the subject in need thereof has venous thrombosis and/or arterial thrombosis.

62. The method of claim 58, wherein the subject in need thereof has one or more of deep vein thrombosis, a pulmonary embolism, an arterial embolism, femoral vein thrombosis, Paget-Schroetter syndrome (PSS), myocardial infarction, superior vena cava thrombosis, jugular vein thrombosis, thrombotic stroke, cerebral venous sinus thrombosis, cavernous sinus thrombosis, retinal vein occlusion, May-Thurner syndrome, portal vein thrombosis, Budd-Chiari syndrome, and renal vein thrombosis.

63. The method of claim 62, wherein the subject in need thereof has thrombotic stroke or cerebral embolism.

64. The method of claim 58, wherein the subject in need thereof is at risk for thrombosis.

65. The method of claim 64, wherein the subject in need thereof is at risk of venous thrombosis and/or arterial thrombosis.

66. The method of claim 64, wherein the subject in need thereof is at risk of one or more of deep vein thrombosis, a pulmonary embolism, an arterial embolism, femoral vein thrombosis, Paget-Schroetter syndrome (PSS), myocardial infarction, superior vena cava thrombosis, jugular vein thrombosis, thrombotic stroke, cerebral embolism, cerebral venous sinus thrombosis, cavernous sinus thrombosis, retinal vein occlusion, May-Thurner syndrome, portal vein thrombosis, Budd-Chiari syndrome, and renal vein thrombosis.

67. The method of claim 66, wherein the subject in need thereof is at risk of thrombotic stroke or cerebral embolism.

68. The method of claim 64, wherein the subject at risk of thrombosis is a subject that has had one or more of a heart valve replacement, a mitral valve repair, and an orthopedic surgery.

69. The method of claim 68, wherein the orthopedic surgery is a surgery that limits mobility.

70. The method of claim 68, wherein the orthopedic surgery is a hip replacement surgery or a knee replacement surgery.

71. The method of claim 64, wherein the subject at risk of thrombosis is a subject that has previously been diagnosed with one or more of a blood clot, a blood clotting disorder, atrial fibrillation, and a cancer.

72. The method of claim 71, wherein the blood clotting disorder is hemophilia, thrombophilia, Von Willebrand disease (VWD), Factor V Leiden, prothrombin gene mutation, antiphospholipid syndrome, and disseminated intravascular coagulation (DIC).

73. The method of claim 71, wherein the cancer is pancreatic cancer, stomach cancer, lung cancer, colon cancer, kidney cancer, or multiple myeloma.

74. The method of claim 64, wherein the subject at risk of thrombosis is a subject that has been immobilized for a prolonged period of time.

75. The method of claim 53, wherein the method comprises treating a cancer, a neurodegenerative disease, an infectious disease, an autoimmune disease, or a metabolic disorder.

76. The method of claim 53, wherein the galloylated polyphenol, or pharmaceutically acceptable salt thereof, is theaflavin 3-gallate, or a pharmaceutically acceptable salt thereof.

77. The method of claim 53, wherein the galloylated polyphenol, or pharmaceutically acceptable salt thereof, is theaflavin 3,3′-digallate, or a pharmaceutically acceptable salt thereof.

78. The method of claim 53, wherein the galloylated polyphenol, or pharmaceutically acceptable salt thereof, is pentagalloyl-glucose, or a pharmaceutically acceptable salt thereof.

79. The method of claim 53, wherein the galloylated polyphenol, or pharmaceutically acceptable salt thereof, is gallic acid, or a pharmaceutically acceptable salt thereof.

80. The method of claim 53, wherein the galloylated polyphenol, or pharmaceutically acceptable salt thereof, is punicalagin, or a pharmaceutically acceptable salt thereof.

81. The method of claim 53, wherein the galloylated polyphenol, or pharmaceutically acceptable salt thereof, is EGCG, or a pharmaceutically acceptable salt thereof.

82. The method of claim 53, wherein the galloylated polyphenol, or pharmaceutically acceptable salt thereof, is catechin 3-gallate, or a pharmaceutically acceptable salt thereof.
